(19) United States
(12) Patent Application Publication
(10) Pub. No.: US 2023/0349901 A1
(43) Pub. Date: Nov. 2, 2023

(54) Methods For Identification of Cognate Pairs of Ligands and Receptors

(71) Applicant: HiFiBio (HK) Limited, Central, Hong Kong (CN)

(72) Inventors: Moustafa Marc Hamze, Paris (FR); Annabelle Patricia Véronique Gérard, Paris (FR)

(21) Appl. No.: 18/002,114

(22) PCT Filed: Jun. 18, 2021
(86) PCT No.: PCT/IB2021/055413

§ 371 Date: Dec. 16, 2022

Related U.S. Application Data
(60) Provisional application No. 62/705,383, filed on Jun. 24, 2020.

(51) Int. Cl. G01N 33/569; G01N 33/68

(52) U.S. Cl. CPC G01N 33/56972; G01N 33/6845; G01N 2500/10

(57) ABSTRACT

Provided herein are methods for identifying cognate pairs of a ligand species and a receptor species. The methods comprise (a) providing a set of ligand species, wherein each ligand species is represented at least one time; (b) providing a set of receptor species, wherein each receptor species is represented at least one time; (c) contacting the set of ligand species with the set of receptor species in a microreactor, wherein upon selective binding of a ligand species with a receptor species an enhanced signal is produced; (d) detecting a cognate pair of ligand species and receptor species by the production of the enhanced signal; and (a) identifying the cognate pair of ligand species and receptor species.

### CROSS REFERENCE TO RELATED APPLICATION

This application claims priority to U.S. Provisional Application No. 62/705,383, filed on Jun. 24, 2020, the disclosure of which is incorporated herein by reference in its entirety.

### FIELD OF THE INVENTION

The invention relates to methods for identifying cognate pairs of ligands and receptors, in particular cognate pairs of T cell receptors and T cell antigens or cognate pairs of B cell receptors and B cell receptor antigens.

### BACKGROUND OF THE INVENTION

Immunotherapy has become an epoch-making and attractive therapeutic modality for cancer, which offers potentially targeted therapy with fewer adverse-effects compared with conventional therapy. One type of immunotherapy is a checkpoint blockade therapy using humanized mAbs specific to CTL antigen 4 (CTLA-4), programmed cell death −1 (PD-1), or its ligand PD-L1. This therapy induced remarkable and durable clinical responses in patients with melanoma, lung, renal, and bladder cancers. However, only a subset of patients (between 20 and 30%) responds to such immune checkpoint therapies and only patients suffering from certain types of cancer.

Accordingly, immunotherapy of cancers using vaccine approaches may be relevant in patients that do not respond to such therapies.

However, very few tumor antigens, which can elicit effective and safe T-cell-mediated antitumor immunity in cancer patients are known. Indeed, such effective tumor antigens need to be overexpressed in cancer tissues, not expressed in normal tissues and be capable of inducing a tumor-antigen specific T-cell response.

Reliable identification of T cell antigens would thus address an unmet need in the field of cancer immunology.

Furthermore, immunotherapy approaches can also potentially be applied to treat autoimmune disease, inflammatory and autoimmune disease, infectious disease and metabolic disease, where efficient and reliable identification of T cell antigens is likewise of great importance.

Autoimmune diseases may also be treated through cell-based therapy or by tolerization approach, which also necessitate reliable identification of antigens of interest.

Unfortunately, despite this potential utility, the discovery and characterization of T cell antigens has moved forward very slowly, in particular because of both the vastness of the T cell repertoire and the large number of potential T cell antigens.

Current methods to identify T cell epitopes generally involve isolating T cells, making individual T cell clones and screening a panel of tumor cell lines or expression libraries from the autologous tumor cells (either fresh or from established cell lines) (Boon et al. (1994) Annu Rev Immunol. 12:337-65). This process is labor intensive and inefficient as both T cell clones and tumor cell lines should be established, which is long and is not possible for all tumor types.

More recently, deep sequencing of the tumor DNA together with RNA analysis has allowed the definition and ranking of candidate epitopes using peptide binding prediction algorithms to the specific MHC alleles avoiding the need of establishing tumor cell lines (Gubin et al. (2015) J. Clin. Invest. 125:3413-3421). However, for MHC class II restricted epitopes recognized by CD4 T cells, the prediction algorithms are not very reliable. Epitopes can also be identified by proteomic analysis of the acid eluate from immuno-precipitate of MHC class I molecules obtained from the tumor cells, further refining the predictive capacity of the process. In both cases, MHC tetramers loaded with the most likely antigen candidate are then synthesized and used to fluorescently label and isolate potential reactive T cells (Yadav et al. (2014) Nature 515:572-576 and Andersen et al. (2012) Nat. Protoc. 7:891-902).

However, making MHC class II tetramers is still challenging for many epitopes.

Alternatively, T cell clones or cell lines expressing cloned T-cell receptor (TCR) are functionally tested against antigen-presenting cells (APC) loaded with synthetic peptides, expression libraries (Gaugler et al. (1994) J. Exp. Med. 179:921-930) or transduced with mRNA coding for the candidate epitopes (Holtkamp et al. (2006) Blood 108:4009-4017). DNA tagged MHC oligomers technique (Bentzen et al. (2016) Nat. Biotechnol. 34:1037-1045) requires prior knowledge of the candidate antigens and is only applicable at the moment for MHC-I restricted epitopes.

However, each method has disadvantages: making T cell clones is extremely labor intensive as is the screening of the resulting clones for antigen specificity; identifying recurrent TCR and/or tumor reactive TCR without cell expansion from bulk population by deconvolution methods is applicable only if a few TCRs of interest are increased in frequency and enough cells are available; elution of peptides from tumor MHC molecules requires many tumor cells; and bioinformatic analysis of MHC epitopes from genomic data requires strong assumptions about the nature of the epitopes.

Altogether, these methods are low-throughput, are based on multi-step processes often requiring the generation of specific reagents (clones, tumor cell lines, MHC tetramers, mRNA, peptides), and are therefore not adapted for unbiased discovery of MHC class I and class II epitopes that may be generated by other mechanisms than mutations or over-expression (e.g. by post-translational modification [notably phosphorylation, ubiquitination, sumoylation], splicing, insertion/deletions).

There is thus an important need for efficient methods to identify cognate pairs of T cells and T cell antigens, in particular, from subjects suffering from cancer, inflammatory and autoimmune disease, infectious disease, or metabolic disease.

Additionally, B cell repertoires (BCR and antibodies) can be a source of therapeutic products and used as immune response monitoring. Such therapeutics products can be antibodies (and all variants thereof, including bispecific antibodies and nanobodies) and engineered cells (including CAR-T and NK cells, among others), which use the ability of antibodies to link a specific antigen preferentially in a native format. Such antigen can be a natural antigen potentially over expressed in cancer cells, a foreign antigen expressed in cancer cells or in normal cells. Libraries of antigens and/or B cell repertoires are usually screened to identify pairs of antigen/B cell repertoire. Identification of antigen can lead to the identification of potential vaccines, biomarkers, and targets. Identification of B cell repertoire corresponding to specific antigens would benefit from high throughput methods to rapidly identify potent and specific therapeutics.

### BRIEF SUMMARY OF THE INVENTION

Having an understanding of a T cell phenotype response can be used to monitor an immune response from a patient before and/or after treatment. Thus, defining foreign antigen (e.g., T cell antigen, B cell antigen, viral antigen, bacterial antigen, parasitic antigen, etc.) responding T cells and/or B cells can lead to the treatment of the diseases caused by the foreign antigen, resulting in the identification of cognate vaccine epitopes. Further, identification of foreign antigen responsive T cell receptors, which could match anti-tumor T cell receptors, could lead to the selection of T cell receptors with high affinity and specificity.

Linking T cell repertoire, antigen, and phenotype over time is necessary to understand the diversity and heterogeneity of immunogenic antigens as well as the responder T cells, at both a phenotypic and transcriptomic level, over time. This will effectively help to stratify patients based on their immunogenic response at different stages and to develop preventive and treatment vaccines

Cell therapy (e.g., T cell, CAR T cells, Treg, stem cells) is another promising therapy showing healing and beneficial clinical outcomes that require the identification of receptor sequences responding to a specific ligand and the characterization of this interaction and their downstream effects. Indeed, linking phenotype-genotype and ligands-receptor could address these unmet needs and improve the selection of the optimal reacting cells showing the appropriate phenotypic and transcriptomic profile. This linkage and matching are useful to fine tune the choice and to select the optimal cells (with the right receptor such as TCR) showing the highest specific activity.

The present invention arises from the unexpected finding that it is possible to screen rapidly and easily up to thousands, including an unlimited set of antigens, without any a priori selection, of tumor antigens for their capacity to bind and activate T cells and to reliably identify such cognate pairs of T cell antigen and T cell receptors with a low error rate without further confirmation of the identification. Furthermore, the identification method designed can be applied to any type of binding pair of ligands and receptors such as viral antigens and T cell receptors, bacterial antigens and T cell receptors, parasitic antigens and T cell receptors, and B cell antigens and B cell receptors.

Thus, provided herein are methods of identifying a cognate pair of a ligand species and a receptor species. The methods comprise (a) providing a set of ligand species, wherein each ligand species is represented at least one time; (b) providing a set of receptor species, wherein each receptor species is represented at least one time; (c) contacting the set of ligand species with the set of receptor species in a microreactor, wherein upon selective binding of a ligand species with a receptor species an enhanced signal is produced; (d) detecting a cognate pair of ligand species and receptor species by the production of the enhanced signal; and (a) identifying the cognate pair of ligand species and receptor species.

In certain embodiments, each ligand species comprises a barcode sequence. In certain embodiments, each receptor species comprises a barcode sequence. In certain embodiments, each ligand species and/or each receptor species comprises a barcode sequence.

In certain embodiments, each ligand species is expressed by or displayed on the surface of a cell or bead or is expressed or present in a cell free extract or in solution. The ligand species can, for example, be expressed by or displayed on the surface of an antigen-presenting cell. The antigen-presenting cell can, for example, be selected from a macrophage, a dendritic cell, a Langerhans cell, a B cell, a monocyte derived dendritic cell, or another cell expressing a MHC class I or II molecule.

In certain embodiments, each receptor species is expressed by or displayed on the surface of a cell or bead or is expressed or present in a cell free extract or in solution.

In certain embodiments, the microreactor is selected from an aqueous droplet, a microcapsule, a microbead, a compartment of a microfluidic chip, or a well (e.g., a well of a tissue culture plate).

In certain embodiments, the signal is selected from a morphological change of any one of a cell, a ligand, or a receptor; a fluorescent signal enhancement; a modification of a fluorescent signal by using a caged compound or by a quenching reaction; a light absorption; a visible structure modification/creation; or a combination of signals thereof. The signal can be a dynamic (on/off, versus on; versus off) and spatio-temporal change. The modification of a fluorescent signal by a quenching reaction can, for example, comprise a FRET, FLIP, FRAP, FLAP, BRET, or FLIM quenching reaction. The morphological changes can be a cell-cell interaction (e.g., like an immunological synapse), a change of cell size, a change of cell granularity, a polarization of peptide/protein localization, or a transfer of material from cell to cell (e.g., proteins, nucleic acids, lipids, and/or carbohydrates)

In certain embodiments, identifying the cognate pair of ligand species and receptor species comprises amplifying the ligand species and the receptor species, wherein at least one of the amplified ligand species and receptor species are sequenced for identification.

In certain embodiments, the set of ligand species can, for example, be selected from T cell antigens, B cell antigens, viral antigens, bacterial antigens, parasitic antigens, neoantigens, tumor associated antigens (TAAs), tumor specific antigens, immune checkpoint molecules, cytokines, carbohydrates, members of the immunoglobulin superfamily, selectins, chemokines, hormone, growth factors, G-protein coupled receptor ligands, or enzyme substrates.

In certain embodiments, the set of receptor species can, for example, be selected from T cell receptors, B cell receptors, immune checkpoint receptors, cytokine receptors, selectins, integrins, members of the immunoglobulin superfamily, cadherins, chemokine receptors, hormone receptors, growth factor receptors, G-protein coupled receptors (GPCRs), or enzymes.

In certain embodiments, the ligand species is a T cell antigen and the receptor species is a T cell receptor, and upon selective binding of the T cell antigen with the T cell receptor, the enhanced signal is produced, wherein the enhanced signal produced is the result of T cell activation.

In certain embodiments, the ligand species is a viral antigen and the receptor species is a T cell receptor, and upon selective binding of the viral antigen with the T cell receptor, the enhanced signal is produced, wherein the enhanced signal produced is the result of T cell activation.

In certain embodiments, contacting the set of ligand species with the set of receptor species in a microreactor occurs for about 0.001 hour to about 8 hours. In certain embodiments, contacting the set of ligand species with the set of receptor species in a microreactor occurs for at least about 8 hours, e.g., about 8 hours to about 48 hours.

In certain embodiments, when the contacting step occurs for about 0.1 hour to about 8 hours, and the ligand species and the receptor species bind with high affinity, the enhanced signal produced is an early marker for T cell activation. In certain embodiments, when the contacting step occurs for about 0.1 hour to about 8 hours, and the ligand species and the receptor species bind with high affinity, the enhanced signal produced is a late marker for T cell activation.

In certain embodiments, when the contacting step occurs for at least about 8 hours, and the ligand species and the receptor species bind with high affinity, the enhanced signal produced can be an early marker or a late marker for T cell activation.

In certain embodiments, when the contacting step occurs for at least about 8 hours, and the ligand species and the receptor species bind with low affinity, the enhanced signal produced is an early marker for T cell activation. Extending the contacting step for the ligand species and the receptor species binding with low affinity can eventually result in an enhanced signal produced by a late marker for T cell activation.

In certain embodiments, the early marker for T cell activation can be, but is not limited to, CD69, CD107a, or a transferrin receptor.

In certain embodiments, the late marker for T cell activation can be, but is not limited to, CD137, HLA-DR, VLA1, PTA1, CD71, CD27, PD-1, TIM3, LAG3, or CTLA4.

In certain embodiments, the signal is detected with an anti-CD69 antibody, an anti-CD107a antibody, an anti-transferrin receptor antibody, anti-CD137 antibody, an anti-HLA-DR antibody, an anti-VLA1 antibody, an anti-PTA1 antibody, an anti-CD71 antibody, an anti-CD27 antibody, an anti-PD1 antibody, an anti-TIM3 antibody, an anti-LAG3 antibody, or an anti CTLA4 antibody.

In certain embodiments, the ligand species is a B cell antigen and the receptor species is a B cell receptor, and upon selective binding of the B cell antigen with the B cell receptor, the enhanced signal is produced. The enhanced signal produced can, for example, be the result of B cell activation, B cell (receptor) specific antigen detection, or target cell activation.

In certain embodiments, the signal is detected with an anti-CD138 antibody, an anti-CD19 antibody, an anti-CD45R antibody, an anti-CD45 antibody, an activation of fluorescent reporter expression, or an inhibition of fluorescent reporter expression.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows the titration of anti-CD137 antibody in droplets. Different concentrations of antibody were co-flowed and incubated with polyclonal preactivated T cells in the droplet overnight.

FIG. 2 shows a schematic overview of T cell activation by antigen presenting cells (APCs) in droplet workflow. K562 antigen presenting cells pulsed with peptide were resuspended in co-flow containing anti-CD137 antibody then co-flowed in a different inlet than the second one containing the T cell clone specific to EBV peptide.

FIG. 3 shows T cell activation in a droplet. K562 pulsed with peptide were resuspended in co-flow containing anti-CD137 antibody then co flowed separately with T cell clone specific to EBV peptide.

FIG. 4 shows images of antigen presenting cell (APC, K562)-T cell interaction in droplets. The upper row shows micrographs of droplets co-encapsulating T cell and K562 loaded with peptide after overnight incubation at 37° C. The others are the fluorescence signal associated as indicated. These binding events result in the red fluorescent signal; anti-CD137 antibody was concentrated on T cells (yellow) instead of being distributed homogeneously throughout the volume of the droplet or on antigen presenting cells (violet) (indicated with arrow).

FIG. 5 shows representative fluorescence plots of IFN-γ secretion in droplet. T cells were co-cultured in droplet with mRNA transfected Antigen Presenting Cell (APC; K562 cells) overnight in presence of anti IFN-γ conjugated antibody (as exemplified here with PE fluorescent dye couple to the antibody). IFN-γ release was detected in the droplet in an indirect ELISA assay using an anti-CD45 bispecific antibody pre-loaded to the T cells and the co-flowed anti-IFN-γ secondary antibody PE-coupled.

FIG. 6 shows a representative workflow for sequence recovery of activated T cells by APC using the Cell-Cap system. Enriched droplet (from sorted droplets containing cells having phenotype of interest) collected in individual micro-wells (optionally checked under microscopy) and then fused with droplets containing hydrogel beads conjugated with cell barcode and gene specific primers. Gene specific primers have been designed to capture TCR, antigen information, and optionally, additional set of genes. The recovered sequences with same cellular barcode would then derive from the same droplet corresponding to the appropriate pair T cell-APC, thus sequences were recovered for TCR and cognate antigen.

FIG. 7 shows linking antigen with TCR sequences: sequences recovered from same droplet showing same cellular barcode and both TCR and antigen (referred to as TMG). As an example, cellular barcode is made of a series of 4 specific set of 11-mers indexes separated by a 4-mers linker). These sequences, blasted against public or private database, matched with TCR α (SEQ ID NO: 1) and β (SEQ ID NO:2) sequences and with the sequences of TMG (SEQ ID NO:3) (tandem minigene corresponding to the transfected antigen into APC). UMI (unique molecular identifiers) is used to quantify transcript produced per cell and is optional.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms used herein have the meanings as set in the specification. All patents, published patent applications, and publications cited herein are incorporated by reference as if set forth fully herein.

Generally, nomenclatures utilized in connection with, and techniques of, cell and tissue culture, molecular biology, and protein and oligo- or polynucleotide chemistry and hybridization described herein are those well-known and commonly used in the art.

Standard techniques are used for recombinant DNA, oligonucleotide synthesis and tissue culture. Enzymatic reactions and purification techniques are performed according to manufacturer's specifications or as commonly accomplished in the art or as described herein.

It must be noted that as used herein and in the appended claims, the singular forms “a,” “an,” and “the” include plural reference unless the context clearly dictates otherwise.

Unless otherwise stated, any numerical value, such as a concentration or a concentration range described herein, is to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the term “nucleic acid” generally refers to at least one molecule or strand of DNA or RNA, comprising at least one nucleobase, such as, for example, a naturally occurring purine or pyrimidine base found in DNA (e.g., adenine “A,” guanine “G,” thymine “T,” and cytosine “C”) or RNA (e.g., A, G, uracil “U,” and C).

“RNA” refers herein to functional RNA, such as mRNA, tRNA, ncRNA, lncRNA, miRNA, siRNA, piRNA, gRNA, telomerase RNA component, RNAi, CRISPR RNA, circular RNA, enhancer RNA, snoRNA, snRNA and rRNA.

As it will be understood by those skilled in the art, the depiction of a single strand also defines the sequence of the complementary strand. Thus, a nucleic acid also encompasses the complementary strand of a depicted single strand. The term nucleic acid thus encompasses complementary DNA. As it will also be appreciated by those skilled in the art, many variants of a nucleic acid may be used for the same purpose as a given nucleic acid. Thus, a nucleic acid also encompasses substantially identical nucleic acids and complements thereof. As it will also be understood by those skilled in the art, a single strand nucleic acid, such as, a primer, may hybridize to the target sequence under hybridization conditions, preferably stringent hybridization conditions. Thus, a nucleic acid also encompasses a primer that hybridizes under hybridization conditions to a target sequence.

The term “barcoded primer” refers to at least one molecule of about 20 to about 200 nucleobases in length that can function to prime nucleic acid synthesis. In particular, the barcoded primer may be of about 30 to about 150 nucleobases in length, of about 40 to about 100 nucleobases in length, of about 50 to about 90 nucleobases in length, of about 60 to about 80 or 70 nucleobases in length. More particularly, in the context of the invention, a barcoded primer is an oligonucleotide comprising a barcode sequence or barcode set of sequences and a primer sequence, wherein each different primer sequence defines a different specificity of barcoded primer. In one embodiment, the barcoded primer comprises from 5′ to 3′ a universal primer sequence, a barcode sequence or barcode set of sequences and a primer sequence.

These definitions refer to at least one single-stranded molecule, but in some embodiments encompass also at least one additional strand that is partially, substantially or fully complementary to the at least one single-stranded molecule. Accordingly, in some embodiments said definitions refer to double stranded molecules.

Thus, in one embodiment, a nucleic acid refers to at least one double-stranded molecule that comprises one or more complementary strand(s) or “complement(s)” of a particular sequence comprising a strand of the molecule.

The “barcode sequence” herein refers to a unique nucleic acid sequence that can be distinguished by its sequence from another nucleic acid sequence, thus permitting to uniquely label a nucleic acid sequence so that it can be distinguished from another nucleic acid carrying another barcode sequence.

In one embodiment, the barcode sequence uniquely identifies the nucleic acids contained in a particular microreactor from nucleic acids contained in other microreactors, for instance, even after the nucleic acids are pooled together.

In some embodiments, the barcode sequence may be used to distinguish tens, hundreds, or even thousands of nucleic acids, e.g., arising from cells contained in different microreactors.

In one embodiment, the barcode sequence may be of any suitable length. The barcode sequence is preferably of a length sufficient to distinguish the barcode sequence from other barcode sequences. In one embodiment, a barcode sequence has a length of 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 72, 74, 76, 78, 80, 85, 90 or more nucleotides, such as 50 to 85, 60 to 80, 70 to 80 nucleotides.

In one embodiment, the barcode sequence consists of more than one barcode sequence, wherein the barcoded sequences are different. Such barcode sequence is called herein “set of barcode sequences.”

In a related embodiment, the different barcode sequences may be taken from a “pool” of potential barcode sequences. If the barcode sequence consists of more than one barcode sequence, the barcode sequences may be taken from the same, or different pools of potential barcode sequences. The pool of sequences may be selected using any suitable technique, e.g., randomly, or such that the sequences allow for error detection and/or correction, for example, by being separated by a certain distance (e.g., Hamming distance) such that errors in reading of the barcode sequence can be detected, and in some cases, corrected. The pool may have any number of potential barcode sequences, e.g., at least 100, at least 300, at least 500, at least 1,000, at least 3,000, at least 5,000, at least 10,000, at least 30,000, at least 50,000, at least 100,000, at least 300,000, at least 500,000, at least 1,000,000, at least 10,000,000, or at least 100,000,000 barcode sequences.

Methods to join different barcode sequences taken from one “pool” or more than one “pool” are known to a person skilled in the art, and include, but are not limited to, the use of ligases and/or using annealing or a primer extension method.

In one embodiment, the barcode sequence is a double stranded or single stranded nucleic acid, or a partially single and double stranded nucleic acid.

A “primer sequence” is typically a short single-stranded nucleic acid, of between 10 to 50 nucleotides in length, designed to perfectly or almost perfectly match a nucleic acid of interest, to be captured and then amplified (e.g., by PCR) or reverse transcribed (e.g., by RT). The primer sequences are “specific” to the nucleic acids they hybridize to, i.e., the primer sequences preferably hybridize under stringent hybridization conditions, more preferably under highly stringent hybridization conditions, and are complementary to or almost complementary to the nucleic acids they hybridize to, also called target sequence.

Typically, the primer sequence serves as a starting point for nucleic acid synthesis, allowing polymerase enzymes such as nucleic acid polymerase to extend the primer sequence and replicate the complementary strand. A primer sequence may be complementary to and hybridize to a target nucleic acid. In some embodiments, a primer sequence is a synthetic primer sequence. In some embodiments, a primer sequence is a non-naturally-occurring primer sequence. A primer sequence typically has a length of 10 to 50 nucleotides. For example, a primer sequence may have a length of 10 to 40, 10 to 30, 10 to 20, 25 to 50, 15 to 40, 15 to 30, 20 to 50, 20 to 40, or 20 to 30 nucleotides. In some embodiments, a primer sequence has a length of 18 to 24 nucleotides.

In one embodiment, the primer sequence is located on the 3′ side of the barcoded primer used in context with the invention (i.e., the primer is in a 3′ position compared to the barcode sequence).

“Gene,” as used herein, can refer to a genomic gene comprising transcriptional and/or translational regulatory sequences and/or a coding region and/or non-translated sequences (e.g., introns, 5′- and 3′-untranslated sequences). The coding region of a gene can be a nucleotide sequence coding for an amino acid sequence or a functional RNA, such as tRNA, rRNA, catalytic RNA, siRNA, miRNA, antisense RNA, lncRNA and piRNA. A gene can also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

The terms “stringent condition” or “high stringency condition,” as used herein, correspond to conditions that are suitable to produce binding pairs between nucleic acids having a determined level of complementarity, while being unsuitable to the formation of binding pairs between nucleic acids displaying a complementarity inferior to said determined level. Stringent conditions are the combination of both hybridization and wash conditions and are sequence dependent. These conditions can be modified according to methods known from those skilled in the art (Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays,” Elsevier, New York). Generally, high stringency conditions are selected to be about 5° C. lower than the thermal melting point (Tm), preferably at a temperature close to the Tm of perfectly base-paired duplexes (Anderson, M. L. M. (1999) Nucleic acid hybridization. New York: Bios Scientific Publisher p. 54). Hybridization procedures are well known in the art and are described for example in Ausubel, F. M., Brent, R., Kingston, R. E., Moore, D. D., Seidman, J. G., Smith, J. A., Struhl, K. eds. (1998) Current protocols in molecular biology. V. B. Chanda, series ed. New York: John Wiley & Sons.

High stringency conditions typically involve hybridizing at about 40° C. to about 68° C., wherein said temperature typically corresponds to the highest melting temperature T_{M} of the nucleic acid to be hybridized with a target sequence, for example, in 5×SSC/5×Denhardt's solution/1.0% SDS, and washing in 0.2×SSC/0.1% SDS at about 60° C. to about 68° C.

As used herein, the term “tissue” refers to a population of cells, generally consisting of cells of the same kind that perform the same, or a similar, function. A tissue can be part of an organ or bone or it can be a loose association of cells, such as cells of the immune system. The tissue can be a healthy tissue or a diseased tissue. In particular, the tissue can be a cancerous tissue or a tissue surrounding a tumor.

As used herein, a “subject” is a mammal, such as a human, but can also be another animal such as a dog, a cat, a cow, a sheep, a pig, a horse, a monkey, a rat, a mouse, a rabbit, a guinea pig etc. Preferably, the subject is a human.

In a particular embodiment, the subject suffers from a disease, in particular from cancer, inflammatory and autoimmune disease, infectious disease or metabolic disease.

By “cancer” is meant herein a class of diseases involving neoplasia which include both cancers that involve a solid tumor and those that do not involve a solid tumor (e.g., leukemia).

By “autoimmune disease” is meant herein a wild range of degenerative diseases caused by the immune system attacking a person's own cells.

By “inflammatory and autoimmune disease” is meant herein a disease first induced by an inflammatory process, initiated by the activation of T cells by antigen-presenting cells, which subsequently leads to the activation of other inflammatory cells and in turn the release of pro-inflammatory cytokines, chemotactic agents and matrix degrading enzymes. Examples of inflammatory and autoimmune diseases are well-known from the person skilled in the art and include rheumatoid arthritis, osteoarthritis, osteoporosis, Crohn's disease, ulcerative colitis, multiple sclerosis, periodontitis, gingivitis, graft versus host reactions, psoriasis, scleroderma, alopecia, Sjogren's syndrome, polymyosititis, pempligus, uveititis, Addison's disease, atopic dermatitis, asthma, systemic lupus erythematosus (SLE), nephropathy and chronic obstructive pulmonary disease (COPD), diabetic retinopathy, and age-related macular degeneration.

By “infectious disease” is meant herein a disease caused by the transmission of a microorganism. In the context of the invention, the term “microorganism” refers equally to viruses, in particular viruses which have a lipid envelope (e.g., an influenza virus), bacteria, parasites, and fungi.

By “metabolic disease” is meant herein any type of disorders in which metabolic errors and imbalances occur and in which the metabolic processes take place in a sub-optimal manner. In a preferred embodiment, the metabolic disease is selected from the group consisting of hyperglycemia, diabetes, in particular type 2 diabetes, obesity, dyslipidemia and hypercholesterolemia. In a particular embodiment, said metabolic disease is diabetes, more particularly type 2 diabetes.

In the context of the invention, the term “cognate pair” of ligands and receptors refers to the pair of a ligand species and the receptor species to which it selectively binds.

By “selectively binding” is meant herein that one member of the pair recognizes and binds to the other member of the pair with greater affinity than to a member of another pair.

By “specifically binding” is meant herein that one member of the pair recognizes and binds to the other member of the pair and has no detectable binding activity for a member of another pair.

As used herein, the term “high affinity” refers to the selective or specific binding of a ligand species to a receptor species, wherein the binding is at a level equal to or less than 3 μM.

As used herein, the term “low affinity” refers to the selective or specific binding of a ligand species to a receptor species, wherein the binding is at a level equal to or greater than 3 μM.

As used herein, the term “ligand species” refers to a member of a particular recognition pair, which selectively binds to, preferably specifically binds to, the second member of said particular recognition pair (or cognate pair).

As used herein, the term “receptor species” refers to a member of a particular recognition pair, which is selectively bound by, preferably specifically bound to, the second member of said particular recognition pair (or cognate pair).

As such, a molecule that is a ligand can also be a receptor and, conversely, a molecule that is a receptor can also be a ligand since ligands and receptors are defined as binding partners.

As used herein, the term “set of ligands” refers to at least one ligand species, preferably a plurality of ligand species, in particular a plurality of ligand species wherein at least two of the plurality of ligands species are part of distinct recognition pairs. Preferably, the set of ligands used in the context of the invention comprises redundant ligand species, i.e. ligand species which are present in the set in multiple copies.

As used herein, the term “set of receptors” refers to at least one receptor species, preferably a plurality of receptor species, in particular a plurality of receptor species wherein at least two of the plurality of receptor species are part of distinct recognition pairs. Preferably, the set of receptors used in the context of the invention comprises redundant receptor species, i.e. receptor species that are present in the set in multiple copies.

In certain embodiments, the set of receptors is expressed by, or displayed on the surface of a cell (or cells), a bead, in particular engineered APC-like beads as disclosed in Neal et al. (2017) J. Immunol. Res. Ther. 2:68-79, or in vitro encoded (i.e., expressed or present in a cell free extract or in solution), as disclosed in Grubaugh et al. (2013) Vaccine 31:3805-3810. Preferably, the set of receptors is expressed by or displayed on the surface of a cell (or cells), one cell or bead expressing or displaying a unique receptor species from the set of receptors.

In another particular embodiment, the set of ligands is expressed by, or displayed on the surface of a cell (or cells), a bead, in particular engineered APC-like beads as disclosed in Neal et al. (2017) J. Immunol. Res. Ther. 2:68-79, or in vitro encoded (i.e., expressed or present in a cell free extract or in solution), as disclosed in Grubaugh et al. (2013) Vaccine 31:3805-3810. Preferably, the set of ligands is expressed by, or displayed on the surface of a cell (or cells), one cell expressing or displaying one ligand species from the set of ligands or multiple distinct ligand species from the set of ligands, preferably between 2 to 1000, between 5 to 900, between 10 to 800, between 20 to 700, between 30 to 600, between 40 to 500, between 50 to 400 distinct ligand species from the set of ligands, in particular 100 to 350, 150 to 300, or 200 to 250 distinct ligand species from the set of ligands.

In a particularly preferred embodiment, the set of receptors is expressed by, or displayed on the surface of a cell (or cells) and the set of ligands is expressed by, or displayed on the surface of another cell (or other cells). Still preferably, the set of receptors is expressed by, or displayed on the surface of a cell (or cells), each cell expressing or displaying a unique receptor species from the set of receptors, and the set of ligands is expressed by, or displayed on the surface of another cell (or other cells), each cell expressing or displaying multiple distinct ligand species from the set of ligands.

In certain embodiments, the receptors can, for example, be T cell receptors (TCR, from a TCR/T cell antigen recognition pair including TCR from a TCR/viral antigen recognition pair), B cell receptors (from a B cell receptor/B cell antigen recognition pair), receptors for stimulatory immune checkpoint molecules (e.g. OX40L from an OX40L/OX40 pair), receptors for inhibitory immune checkpoint molecules (e.g. PD-L1 from a PD-L1/PD-1 pair), cytokine receptors (from a cytokine/cytokine receptor pair), selectins (from a selectin/carbohydrate pair), integrins (from an integrin/member of the immunoglobulin superfamily pair), members of the immunoglobulin superfamily (from a member of the immunoglobulin superfamily/selectin pair, or from a pair comprising two members of the immunoglobulin superfamily), cadherins (from a pair comprising two cadherins), chemokine receptors (from a chemokine/chemokine receptor pair), hormone receptors (from an hormone/hormone receptor pair), growth factor receptors (from a growth factor/growth factor receptor pair), G protein-coupled receptors (GPCR, from a GPCR/corresponding ligand pair) or enzymes (from an enzyme/corresponding substrate pair).

In a preferred embodiment, the set of receptors is a set of T cell receptors.

In certain embodiments, the ligands can, for example, be T cells antigens (from a TCR/T cell antigen recognition pair), B cell antigen (from a B cell receptor/B cell antigen recognition pair), viral antigens, bacterial antigens, parasitic antigens, neoantigens (i.e., antigens which result from gene mutations or aberrant expression in tumor cells and whose expression is uniquely found in tumor cells), tumor associated antigens (TAAs), tumor specific antigens, stimulatory immune checkpoint molecules (e.g. OX40 from an OX40L/OX40 pair), inhibitory immune checkpoint molecules (e.g. PD-1 from a PD-L1/PD-1 pair), cytokines (from a cytokine/cytokine receptor pair), carbohydrates (from a selectin/carbohydrate pair), members of the immunoglobulin superfamily (from a pair comprising two members of the immunoglobulin superfamily), selectin (from a member of the immunoglobulin superfamily/selectin pair), chemokines (from a chemokine/chemokine receptor pair), hormones (from an hormone/hormone receptor pair), growth factors (from a growth factor/growth factor receptor pair), ligands of GPCRs (from a GPCR/corresponding ligand pair) or substrates (from an enzyme/corresponding substrate pair). In a preferred embodiment, the set of ligands is a set of T cell antigens (peptides, glycolipids or small metabolites such as 5-A-RU derivatives), preferably bound to major histocompatibility complex (MHC molecules that can be class I, class II or MR1) or to CD1a, b, c, or d molecules.

Foreign antigens, such as, viral antigens, bacterial antigens, or parasitic antigens can, for example, include, but are not limited to, a viral antigen, bacterial antigen, or parasitic antigen selected from at least one of the following organisms: Borrelia bacteria (e.g., Borrelia burgdorferi), Chikungunya Virus (CHIKV), Chlamydia bacteria (e.g., Chlamydia trachomatis), Cytomegalovirus (CMV), Dengue Virus (DENV), Ebola Virus (EVD), E. coli (e.g., Shiga-Like toxin), Epstein Barr Virus (EBV), Feline Leukemia Virus, Hantavirus, Hepatitis Virus (e.g., Hepatitis A, B, C, D, and/or E virus), Herpes Virus, Helicobacter pylori, Human endogenous retrovirus K (HERV-K), Human Immunodeficiency Virus (HIV), Human T-cell Leukemia Virus (HTLV), Influenza Virus, Lassa Virus, Plasmodium parasites (e.g., which cause malaria), Mumps Virus (e.g., Mumps orthorubulavirus), Mycoplasma bacteria, Norovirus, Papillomavirus (HPV), Parvovirus, Rhinovirus, Rotavirus, Rubella virus, Salmonella bacteria (e.g., Salmonella typhi), SARS coronavirus (SARS-CoV), Toxoplasma parasite (e.g., Toxoplasma gondii), Treponema bacteria (e.g., Treponema pallidum, Treponema carateum), Trypanosoma parasite (e.g., Trypanosoma cruzi, which causes Chagas disease), Varicella Zoster Virus (VZV), Variola Virus, West Nile Virus (WNV), and/or Zika virus (ZIKV). Viral antigens are known to have stronger TCR affinity, see, e.g., Aleksic et al., Eur. J. Immunol. 42(12):3174-9 (2012), which could lead to higher enhanced signal detection in the methods disclosed herein. Foreign antigens are known to those skilled in art, see, e.g., Medical Microbiology, 4^{th} edition, Chapter 6: Normal Flora; Baron S., editor; Galveston, TX; University of Texas Medical Branch at Galveston (1996); Laufer et al., “Microbial communities of the upper respiratory tract and otitis media in children,” mBio 2(1):e00245-10 (2011).

### T Cell Antigen/T Cell Receptor

In a particular embodiment, the set of receptors is a set of T cell receptors, preferably displayed on the surface of T cells, each T cell preferably having a unique T cell receptor, and the set of ligands is a set of T cell antigens, preferably bound to major histocompatibility complex (MHC) typically displayed on the surface of antigen-presenting cells (APCs), each APC preferably displaying multiple antigen species.

By “T cell antigen” is meant herein a CD4⁺ T cell antigen or a CD8⁺ T cell antigen. A “CD4⁺ T cell antigen” refers to any antigen that is recognized by and triggers an immune response in a CD4⁺ T cell, e.g., an antigen that is specifically recognized by a T cell receptor on a CD4⁺ T cell via presentation of the antigen or portion thereof bound to a Class II major histocompatibility complex molecule (MHC). A “CD8⁺ T cell antigen” refers to any antigen that is recognized by and triggers an immune response in a CD8⁺ T-cell, e.g., an antigen that is specifically recognized by a T cell receptor on a CD8⁺ T cell via presentation of the antigen or portion thereof bound to a Class I major histocompatibility complex molecule (MHC). T cell antigens are generally proteins or peptides but may be other molecules such as lipids and glycolipids and any derivatives thereof.

Tetramers, multimers, and derivatives thereof, where the antigen specificity is carried by a barcode, or by the gene are also contemplated. The tetramer, or any derivative can be synthesized in the droplet by in vitro transcription translation (IVTT), including the antigen and the corresponding vector. Such a vector can include genes encoding for the expression of a soluble TCR.

Preferably, the set of ligands is a set of T cell antigens bound to major histocompatibility complex (MHC) displayed on the surface of antigen-presenting cells (APCs).

In the context of the invention, the term “antigen-presenting cells” or “APCs” encompass a heterogeneous group of immunocompetent cells that mediate the cellular immune response by processing and presenting antigens to the T cells. Antigen-presenting cells include, but are not limited to macrophages, dendritic cells, Langerhans cells, B cells, monocyte derived dendritic cell, artificial APCs, engineered APC, or other cells expressing MHC class I molecule or MHC class II molecules.

The APCs can be B cells, in particular immortalized B cells such as Epstein-Barr virus (EBV)-immortalized B cells.

In a particular embodiment, the APCs are autologous immortalized B cells from a subject of interest, as defined above, or heterologous immortalized B cells carrying the same MHC as the subject of interest, as defined above.

By “heterologous B cells carrying the same MHC as the subject of interest” is meant herein B cells which do not originate from the subject of interest, but which carry the same MHC as the subject of interest.

By “MHC” or “major histocompatibility complex” is meant herein a complex of genes (and the molecules encoded by them) that encode cell-surface molecules required for antigen presentation to T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an a chain encoded in the MHC associated noncovalently with β2-microglobulin. Class I MHC molecules are expressed by nearly all nucleated cells and have been shown to function in antigen presentation to CD8⁺ T cells. Class I molecules include HLA-A, -B, and -C in humans. Class II MHC molecules also include membrane heterodimeric proteins consisting of noncovalently associated α and β chains. Class II MHC are known to interact with CD4⁺ T cells and, in humans, include HLA-DP, -DQ, and DR. The term “MHC restriction” refers to a characteristic of T cells that permits them to recognize antigen only after it is processed and the resulting antigenic peptides are displayed in association with either a class I or class II MHC molecule. Methods of identifying and comparing MHC are well known in the art and are described in Allen et al. (1994) Human Imm. 40:25-32; or Santamaria et al. (1993) Human Imm. 37: 39-50.

In a particular embodiment, each APC expresses or displays at least one T cell antigen, preferably multiple distinct T cell antigens, from the set of T cell antigens, preferably between 2 to 1000, between 5 to 900, between 10 to 800, between 20 to 700, between 30 to 600, between 40 to 500, between 50 to 400 distinct T cell antigens from the set of T cell antigens, in particular 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 distinct T cell antigens from the set of T cell antigens. In a particular embodiment, each APC expresses or displays between 10 to 1000, more preferably, 300 distinct T cell antigen species.

In a particular embodiment, the set of APCs displaying at least one T cell antigen, in particular multiple distinct T cell antigens, is obtained by introducing a library of nucleic acids encoding T cell antigens obtained from a tissue of a subject of interest, as defined above, into autologous APCs from the subject of interest, or heterologous APCs carrying the same MHC as the subject of interest, as defined above.

In another particular embodiment, the set of APCs displaying at least one T cell antigen, in particular multiple distinct T cell antigens, is obtained by introducing into APCs a library of synthetic mRNAs (either as a tandem gene or as single gene) encoding antigens, said mRNAs being identified by sequencing the genome, exome, or transcriptome of a tumor and generated by in vitro-transcription.

In another particular embodiment, the set of APCs displaying at least one T cell antigen, in particular multiple distinct T cell antigens, is obtained by introducing into APCs a library of synthetic mRNAs (either as a tandem gene or as single gene) encoding antigens, said mRNAs being identified by sequencing the genome, exome, or transcriptome of a tumor and generated by split and pool with the individual mRNA.

In another particular embodiment, the set of APCs displaying at least one T cell antigen, in particular multiple distinct T cell antigens, is obtained by introducing into APCs a library of synthetic mRNAs (either as a tandem gene or as single gene) encoding antigens, said mRNAs being identified by sequencing the genome, exome, or transcriptome of a tumor and generated by using penetration based delivery of DNA allowing mRNA translation.

In another particular embodiment, the set of APCs displaying at least one T cell antigen, in particular multiple distinct T cell antigens, is obtained by introducing into APCs individual DNA (either as a tandem gene or as single gene encoding antigens, said antigen being identified by sequencing the genome, exome, or transcriptome of a tumor) made on beads and transcribed into mRNA and transfected in drop.

In another particular embodiment, the set of APCs displaying at least one T cell antigen, in particular multiple distinct T cell antigens, is obtained by introducing into APCs individual DNA (either as a tandem gene or as single gene encoding antigens, said antigen being identified by sequencing the genome, exome, or transcriptome of a tumor) made on beads and transcribed into mRNA and transfected or transduced in APC.

In another particular embodiment, the set of APCs displaying at least one T cell antigen, in particular multiple distinct T cell antigens, is obtained by introducing known tagged T cell antigens and/or known tagged nucleic acids encoding T cell antigens, into APCs.

By “tagged” is meant herein bearing a tag such as a nucleic acid of known sequence, a fluorescent dye or a label. Typically, the tag may be a barcode sequence as defined above.

In a particular embodiment, the library of nucleic acids encoding T cell antigens is chemically synthesized based on the sequencing of the patient or pathogen RNA or DNA.

In another particular embodiment, the library of nucleic acids encoding T cell antigens is obtained by amplification (e.g., by PCR) of nucleic acids from a tissue of a subject of interest, as defined above.

In a particular embodiment, the library of nucleic acids encoding T cell antigens is a cDNA library. In another embodiment, the library of nucleic acids encoding T cell antigens is an mRNA library.

A library of nucleic acids encoding T cell antigens obtained from a tissue of a subject of interest can be obtained by methods well-known from the skilled person. In particular, cells, for example, MHC-I or MHC-II expressing cells or tumor cells, can be extracted from a tissue of the subject of interest by known techniques such as DNase, protease (such as collagenase) and mechanical digestion. RNAs can be isolated from said cells by techniques well-known to the skilled person such as silica columns or acid phenol techniques. These RNAs can then be reverse transcribed using well-known techniques to obtain a cDNA library. Preferably, the cDNA library is obtained by reverse transcription using primers which hybridize with RNAs. The primers can either prime on all mRNAs by hybridizing to the poly(A) tail (anchored-oligo-dT primers) or be designed to prime only on a specific subset of RNAs or to prime randomly to any RNA.

In a particular embodiment, said library is normalized to reduce biases in the library due to differences in mRNA concentrations.

A range of normalization techniques are well-known to the skilled person, such as duplex-specific nuclease (DSN)-based normalization of cDNA libraries (Bogdanov et al. (2010). Curr. Protoc. Mol. Biol. Chapter 5:Unit 5.12.1-27), and normalization of cDNA libraries by mRNA-cDNA hybridization and subtraction (Chen (2003) In S. Y. Ying (Ed.), Generation of cDNA Libraries: Methods and Protocols (pp. 33-40) Totowa, NJ: Humana Press).

In a particular embodiment, the library of nucleic acid encoding T cell antigens contain universal sequences allowing specific amplification and sequencing in subsequent steps of the method of identification of the invention, as defined below.

As used herein, the term “universal sequence” refers to a sequence that can be attached, for example by ligation or any other suitable method (like overlap extension PCR, PCR, primer extension or direct DNA synthesis), to a nucleic acid sequence, particularly in a library of nucleic acid molecules, such that the same sequence is attached to a plurality of different nucleic acid molecules. Such a universal sequence is particularly useful for analyzing multiple samples simultaneously. Examples of universal sequences are universal primers and universal priming sites. A universal priming site contains a “common priming site” to which an appropriate primer can bind to and which can be utilized as a priming site for synthesis of nucleic acid sequences complementary to the nucleic acid sequence attached to the universal primer.

Introduction of a library of nucleic acids encoding T cell antigens into APCs can be carried out by any method well-known from the skilled person, such as by transduction with viral vectors, by electroporation, or by transfection (lipo-, nucleo-fection, nanoparticle based, or using cell-penetrating peptides, for example).

In a particular embodiment, the introduction of the library of T cell antigens-encoding nucleic acids, in particular of the cDNA library of T cell antigens-encoding nucleic acids, into said APCs is performed by transducing said APCs with viral vectors carrying said library.

By “viral vector” is meant herein a virus, or recombinant thereof, capable of encapsulating desirable genetic material and transferring and integrating the desirable genetic material into a target cell, thus enabling the effective and targeted delivery of genetic material both ex vivo and in vivo. Examples of viral vectors include adenovirus vectors, adeno-associated virus vectors, herpes simplex virus vectors, retrovirus vectors, lentivirus vectors, Semliki forest virus vectors, Sindbis virus vectors, vaccinia virus vectors, fowlpox virus vectors, baculovirus vectors and Sendai virus vectors. Preferably, said viral vector is a lentivirus vector.

As will be understood by the skilled person, when the introduction of T cell antigen-encoding nucleic acids into APCs is performed by transduction with viral vectors, the number of distinct T cell antigens expressed or displayed by said APCs depends on the multiplicity of infection (MOI) at which said viral vectors were used to transduce said APCs.

Accordingly, in a particular embodiment, the introduction of the library of T cell antigen-encoding nucleic acids into said APCs is performed by transducing said APCs with viral vectors carrying said library at a multiplicity of infection between 0.01 and 1000, between 0.05 and 900, between 0.1 and 800, between 0.5 and 700, between 1 and 600, between 2 and 500, between 3 and 450, between 4 and 400, between 5 and 350, between 6 and 300, between 7 and 250, between 8 and 200, between 9 and 150, or between 10 and 100, in particular at a multiplicity of infection of 20, 30, 40, 50, 60, 70, 80 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500, thereby generating APCs expressing at least one antigen up to multiple antigens.

In another embodiment, the introduction of the library of T cell antigen-encoding nucleic acids, in particular, of the RNA library of T cell antigens-encoding nucleic acids, into said APCs is performed by transfection of APCs with said mRNAs or corresponding DNAs. Such transfection may typically be carried out by lipofection, nucleo-fection, nanoparticle-based transfection or using cell-penetrating peptides (Rádis-Baptista et al. (2017) Journal of Biotechnology 252:15-26) such as penetratin, that are covalently coupled to the RNA. The same principle applies for the expression of TCRs by autologous or heterologous T cells, or T cell lines to screen for TCR specificity and/or affinity/avidity towards antigen/MHC complex.

The term “T cell receptor” or “TCR” herein refers to an antigen-recognition molecule present on the surface of T cells (i.e., T lymphocytes). This definition expressly includes the understanding of the term as known in the art, and includes, for example, a receptor that comprises or consists of a disulfide-linked heterodimer of the highly variable alpha or beta chains expressed at the cell membrane as a complex with the invariant CD3 chains, or a receptor that comprises or consists of variable gamma and delta chains expressed at the cell membrane as a complex with CD3 on a subset of T cells. The antigen recognition domain of TCRs is typically composed of an alpha chain and a beta chain, or of a gamma chain and a delta chain, encoded by separate genes.

T-cell receptor genes undergo a unique mechanism of genetic recombination, called V(D)J recombination, that occurs only in developing lymphocytes during the early stages of T cell maturation. It results in the highly diverse repertoire of T cell receptors (TCRs) found on T cells.

Preferably, the set of receptors is a set of TCRs displayed on the surface of T cells.

The term “T cells” or “T lymphocytes” as used generically herein may refer to, for example, CD4⁺ helper T cells (e.g., TH1, TH2, TH9 and TH17 cells), CD8⁺ cytotoxic T cells, antigen experienced T cells, naïve T cells, central T cells, effector T cells, CD4⁺ regulatory/suppressor T cells (Treg cells), natural killer T cells, γδ T cells, and/or autoaggressive T cells (e.g., TH40 cells), mucosal associated invariant T cells (MAIT), exhausted T cells, memory T cells, central memory T cells, effector memory T cells, tissue resident T cells.

Preferably, in the set of T cells displaying TCRs, each T cell expresses or displays a unique T cell receptor (TCR).

In a particular embodiment, the T cells of the set of T cells displaying TCRs, are derived from the same subject of interest as the library of nucleic acids encoding T cell antigens, as defined above, used to obtain the set of APCs displaying T cell antigens. In particular, this embodiment facilitates the detection of private T cell antigens.

By “private antigen” is meant herein an antigenic specificity restricted to one or a few individuals.

In another particular embodiment, the T cells of the set of T cells displaying TCRs, are not derived from the same subject of interest as the library of nucleic acids encoding T cell antigens, as defined above, used to obtain the set of APCs displaying T cell antigens. In particular, this embodiment facilitates the detection of public T cell antigens.

By “public antigen” is meant herein an antigen that is present in more than 5% more particularly in more than 10% of a population.

In a particular embodiment, the T cells of the set of T cells displaying TCRs are activating to allow their expansion after being collected from the subject.

### B Cells Receptors/B Cells Antigens

In another particular embodiment, the set of receptors is a set of B cell receptors (BCR or antibodies) and the set of ligands is a set of B cell antigens.

As used herein, the term “BCR” refers to a transmembrane receptor protein located on the outer surface of B cells. The receptor's binding moiety is composed of a membrane-bound antibody that, like most antibodies, has a unique and randomly determined antigen-binding site (see V(D)J recombination). When a B cell is activated by its first encounter with an antigen that binds to its receptor (its “cognate antigen”), the cell proliferates and differentiates to generate a population of antibody-secreting plasma B cells and memory B cells.

The term “antibody” refers to immunoglobulin molecules and immunologically active portions of immunoglobulin molecules, i.e., molecules that contain an antigen binding site which immunospecifically binds an antigen. As such, the term antibody encompasses not only whole antibody molecules, but also antibody fragments as well as variants of antibodies, including derivatives such as humanized antibodies. In certain conventional antibodies, two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA, IgE and IgY. Each chain contains distinct constant region sequences. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity determining regions (CDRs) refer to amino acid sequences which, together, define the binding affinity and specificity of the natural Fv region of a native immunoglobulin binding-site. The light and heavy chains of an immunoglobulin each have three CDRs, designated LCDR1, LCDR2, LCDR3, and HCDR1, HCDR2, HCDR3, respectively. Therefore, an antigen-binding site includes six CDRs, comprising the CDR set from each of a heavy and a light chain V region. Framework Regions (FRs) refer to amino acid sequences interposed between CDRs, i.e., to those portions of immunoglobulin light and heavy chain variable regions that are relatively conserved among different immunoglobulins in a single species, as defined by Kabat, et al. (Sequences of Proteins of Immunological Interest (National Institutes of Health, Bethesda, Md., 1991).

The term antibody further denotes single chain antibodies, for instance Camelidae antibodies, or nanobodies or V_{HH}.

Antibody genes generally undergo a unique mechanism of genetic recombination, called V(D)J recombination, that occurs only in developing lymphocytes during the early stages of B cell maturation. The antibody genes may be further subjected to somatic hypermutation, and the combination of V(D)J recombination and somatic hypermutation results in the highly diverse repertoire of antibodies/immunoglobulins (Igs) found on B cells.

In a particular embodiment, when the set of receptors is a set of B cell receptors, said B cell receptors are displayed by B cells.

### Microreactors and Co-Compartmentalization

By “co-compartmentalizing” ligand species and receptor species is meant herein forming a plurality of microreactors, each microreactor separating a group of ligand species and/or receptor species, preferably a group of at least one ligand species and optionally at least one receptor species, from the remaining ligand species and receptor species provided by the set of ligands and receptors.

In the context of the invention, the co-compartmentalization/microreactor step may be carried out by any suitable method, such as by microfluidics, flow cytometry cell-based sorting, and/or limiting dilution.

In a particular embodiment, the microreactors are wells or microfabricated wells.

In another particular embodiment, the microreactors are aqueous droplets, in particular in a continuous immiscible phase.

A “droplet” generally refers to a measure of volume and further refers in context of the present invention, to an isolated portion of a first fluid that is surrounded by a second fluid. It is to be noted that a droplet is not necessarily spherical, but may assume other shapes as well, for example, depending on the external environment.

Preferably, each droplet has a volume at least equal to the volume of two mammalian cells.

In another particular embodiment, the microreactors are microcapsules. The microcapsules can refer to a measure of volume and further refers in context of the present invention, to an isolated portion of a first coating material that surround a second material. It is to be noted that a microcapsule is not necessarily spherical, but may assume other shapes as well, for example, depending on the external environment. A “microcapsule” generally refers to hollow microparticle composed of a solid shell surrounding a core-forming space available to permanently or temporarily entrapped substances. The substances can be drugs, pesticides, dyes, cells, combinations thereof and similar materials. The solid shell can, for example, enclose solids, liquids, or gases inside a micrometric wall made of hard or soft soluble film. The coating materials generally used for coating are ethyl cellulose, polyvinyl alcohol, gelatin, sodium alginate.

Preferably, each microcapsule has a volume at least equal to the volume of two mammalian cells.

In another particular embodiment, the microreactors are microbeads. The microbeads can refer to a measure of volume and further refers to an isolated portion of a first semi-solid material that is surrounded by a fluid, either permeant or not to the semi-solid bead. It is to be noted that a microbead is not necessarily spherical, but may assume other shapes as well, for example, depending on the external environment. A “microbead” generally refers to a semi-solid porous or not structure, occupying the whole volume available to permanently or temporarily entrapped substances. The substances can be drugs, pesticides, dyes, cells, combinations thereof and similar materials. The semi-solid porous structure can, for example, enclose solids, liquids, or gases inside a micrometric wall made of hard or soft soluble film. The materials generally used for forming microbeads include polymers like agarose, acrylamide, sodium alginate.

Preferably, each microbead has a volume at least equal to the volume of two mammalian cells.

It is understood that cells can be encapsulated in microcapsules or microbeads before the cells achieve their transformation of droplet into microcapsules or microbeads.

The average volume of a mammalian cell is well-known to the skilled person and is typically about 0.002 nL.

In a particular embodiment, each droplet or microcapsule or microbead has a volume of less than 20 nL. In one embodiment, each droplet has a volume of less than 15 nL, less than 10 nL, less than 9 nL, less than 8 nL, less than 7 nL, less than 6 nL, less than 5 nL, less than 3 nL, less than 2.5 nL, less than 2 nL, less than 1.5 nL, less than 1 nL, less than 0.5 nL, less than 0.2 nL, less than 0.1 nL, less than 0.05 nL, for example 20 pL to 3 nL, 30 pL to 1 nL, 40 pL to 500 pL, 50 pL, to 250 pL, 60 pL to 100 pL, or 0.1 nL to 3 nL, 0.5 nL to 3 nL, 1 nL to 3 nL, typically, 0.1 nL, 0.5 nL, 1 nL, 1.2 nL, 1.4 nL, 1.6 nL, 1.8 nL, 2.0 nL, 2.2 nL, 2.4 nL, 2.6 nL, 2.8 nL, 3 nL.

Such droplets or micro-capsule or micro-bead may be prepared by any technique well-known from the skilled person, in particular, by microfluidics technique.

As it will be understood by the skilled in the art and as further explained below, the number of ligands and receptors, in particular of T cell antigen-displaying APCs and TCR displaying T cells, co-compartmentalized in one microreactor, for instance a droplet, follows a probability distribution, for example a Poisson distribution, and depends on, for example, the concentration of the first type of cells in the first fluid, the concentration of the second type of cells in the second fluid, the geometry of the main channel and the secondary channel, the injection parameters of the first fluid, of the second fluid and of the carrier fluid used.

If the ligands and/or receptors are displayed on cells or particles, the distribution of cells or particles, and hence also the distribution of ligands and/or receptors, will typically follow a Poisson distribution. However, if the microreactors are droplets, a variety of microfluidic techniques, familiar to the skilled person, allow distributions other than Poisson distribution, in particular distributions in which a higher fraction of droplets contains single cells/particles. These techniques include methods consisting in ordering the particles/cells before compartmentalization into droplets using inertial forces and mediated by secondary flows such as Dean flow (see Edd et al. (2008) Lab Chip 8:1262-1264; Kemna et al. Lab Chip (2012) 12:2881-2887, Lagus and Edd (2013) RSC Advances 3:20512-20522, Schoeman et al. (2014) Electrophoresis 35:385-392, Schoeman et al. (2018) Scientific Reports 8:3714, US 2013/011210, US 2010/021984 and US 2011/0223314), methods consisting in the isolation/sorting of the droplet containing a single cell/particle or a pair of cells/particles to reduce the number of droplets to analyze/measure (see Hu et al. (2015) Lab Chip 15:3989-3993; Chung et al. (2017) Lab Chip 17:3664-3671 and Shembekar et al. (2018) Cell Reports 22:2206-2215), methods consisting in forcing the cells/particles to flow in a narrow bottleneck to reduce chances of encapsulating multiple cells/particles of the same sample (see Ramji et al. (2014) Biomicrofluidics 8:034104), methods consisting in the production of droplets on demand when a cell/particle is passing in front of the nozzle (see Schoendube et al. (2015) Biomicrofluidics 9:014117; Leibacher et al. (2015) Biomicrofluidics 9:024109 and Yusof et al. (2011) Lab. Chip 11:2447-2454).

Accordingly, in a particular embodiment, a plurality of receptor species, in particular of TCR displaying T cells comprised in an aqueous composition are co-compartmentalized with a plurality of ligands, in particular of antigen-displaying APCs, into a plurality of microreactors, in particular in a plurality of microfluidic droplets, and the number of receptor species, in particular of TCR displaying T cells, co-compartmentalized into one microreactor, in particular co-compartmentalized in one droplet follows, depending on the parameters used, a probability distribution, in particular a Poisson distribution. The parameters can be adapted to obtain, for instance, most microreactors having either 1 or 0 receptor, in particular, a TCR displaying T cell, in it, thus minimizing the number of compartments containing several receptors.

The parameters used to co-compartmentalize receptor species, in particular TCR displaying T cells, with ligand species, in particular antigen-displaying APCs, can be adapted to obtain at least some of the microreactors comprising a single receptor species, in particular a single TCR displaying T cell.

Similarly, the parameters used to co-compartmentalize receptor species, in particular TCR displaying T cells, with ligand species, in particular antigen-displaying APCs, can be adapted to obtain at least some of the microreactors comprising a single ligand species, in particular a single antigen-displaying APC.

In a particular preferred embodiment, a set of microreactors is created, each comprising at least one ligand species, in particular at least one T cell antigen, more particularly at least one T cell antigen-displaying APC, and at least one receptor species, in particular at least one TCR, more particularly at least one TCR displaying T cell.

As will be understood by the skilled person, some microreactors may however be created which do not include any receptor species.

Preferably, a set of microreactors is created, each comprising at least one ligand species, in particular at least one T cell antigen, more particularly at least one T cell antigen-displaying APC, and one or a small number of receptor species, in particular one or a small number of TCR, more particularly one or a small number of TCR displaying T cells.

By “small number” is meant herein less than 10, such as 10, 9, 8, 7, 6, 5, 4, 3, 2 or 1.

In another preferred embodiment, a set of microreactors is created, each comprising one or a small number of ligand species, in particular one or a small number of T cell antigens, more particularly one or a small number of T cell antigen-displaying APC, and at least one receptor species, in particular at least one TCR, more particularly at least one TCR displaying T cell.

In a still preferred embodiment, a set of microreactors is created, each comprising one or a small number of ligand species, in particular one or a small number of T cell antigens, more particularly one or a small number of T cell antigen-displaying APC, and one or a small number of receptor species, in particular one or a small number of TCR, more particularly one or a small number of TCR displaying T cells.

In a preferred embodiment, a set of microreactors is created, each comprising one ligand species, in particular one T cell antigen, more particularly one T cell antigen-displaying APC, and one receptor species, in particular one TCR, more particularly one TCR displaying T cell.

In a particularly preferred embodiment, a set of microreactors is created, each comprising multiple ligand species, in particular multiple T cell antigens, more particularly one APC displaying multiple T cell antigens or multiple APCs displaying single (or multiple) T cell antigens, and one receptor species, in particular one TCR, more particularly one TCR displaying T cell.

Accordingly in one embodiment, the set of microreactors is formed by co-compartmentalization of ligand species, in particular antigen-displaying APCs, and receptor species, in particular TCR-displaying T cells, at a frequency of 1 to 20,000 microreactors per second, such as 1 to 15,000 microreactors per second, 1 to 10,000 microreactors per second, 1 to 9000 microreactors per second, 1 to 8000 microreactors per second, 1 to 7000 microreactors per second, 1 to 6000 microreactors per second, 1 to 5000 microreactors per second, 1 to 4000 microreactors per second, 1 to 3000 microreactors per second, 1 to 2000 microreactors per second, 1 to 1000 microreactors per second, 1 to 800 microreactors per second, 1 to 700 microreactors per second, 1 to 600 microreactors per second, 1 to 500 microreactors per second, 1 to 400 microreactors per second, 1 to 300 microreactors per second, 1 to 200 microreactors per second, 1 to 100 microreactors per second, 1 to 80 microreactors per second, 1 to 70 microreactors per second, 1 to 50 microreactors per second, for example 10 to 300 microreactors per second, 50 to 300 microreactors per second, 100 to 300 microreactors per second, 150 to 300 microreactors per second, 150 to 250 microreactors per second, 175 to 250 microreactors per second, typically, 1 to 1000 microreactors per second, preferably 175 to 250 microreactors per second.

The set of microreactors, in particular the set of microfluidic droplets, may be obtained by any suitable technique. In particular, the set of microreactors may be formed by (a) providing a first fluid source, the first fluid comprising a suspension of a set of receptors as defined above, (b) providing a second fluid source, the second fluid comprising a suspension of a set of ligands as defined above; (c) providing a carrier fluid, the carrier fluid being immiscible with the first fluid and the second fluid, (d) injecting the carrier fluid in a main channel of a chip, (e) generating a flow of microreactors, in particular droplets, in the carrier fluid by injecting the second fluid and the first fluid in at least a secondary channel of the chip, the secondary channel opening in the main channel, each generated microreactor, in particular droplet, comprising a mix of the first fluid and the second fluid, wherein the concentration of the receptors in the first fluid, the concentration of the ligands in the second fluid, the geometry of the main channel and the secondary channel, the injection parameters of the first fluid, of the second fluid and of the carrier fluid are adapted such that each microreactor, in particular droplet, comprises at least one receptor species, preferably only a single receptor species, and at least one ligand species and preferably presents a volume of less than 20 nL.

Alternatively, the set of microreactors may be formed by (a) providing a first fluid source, the first fluid comprising a suspension of a set of pre-formed receptors and ligands as defined above, (b) providing optionally a second fluid source, the second fluid comprising reagents for detection as defined above, (c) providing a carrier fluid, the carrier fluid being immiscible with the first fluid and the second fluid.

In one embodiment, the first and second fluid sources are organized in the form of a junction.

The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, International Patent Application No. PCT/US2004/010903, filed Apr. 9, 2004, entitled “Formation and Control of Fluidic Species,” by Link, et ah, published as WO 2004/091763 on Oct. 28, 2004, or International Patent Application No. PCT/US2003/020542, filed Jun. 30, 2003, entitled “Method and Apparatus for Fluid Dispersion,” by Stone, et ah, published as WO 2004/002627 on Jan. 8, 2004.

In some embodiments, the junction may be configured and arranged to produce substantially monodisperse droplets.

The amount of receptor species/droplet may also be referred to as loading rate. For example, the average loading rate may be less than about one receptor species/droplet, less than about 0.9 receptor species/droplet, less than about 0.8 receptor species/droplet, less than about 0.7 receptor species/droplet, less than about 0.6 receptor species/droplet, less than about 0.5 receptor species/droplet, less than about 0.4 receptor species/droplet, less than about 0.3 receptor species/droplet, less than about 0.2 receptor species/droplet, less than about 0.1 receptor species/droplet, less than about 0.05 receptor species/droplet, less than about 0.03 receptor species/droplet, less than about 0.02 receptor species/droplet, or less than about 0.01 receptor species/droplet. In some cases, lower receptor species loading rates may be chosen to minimize the probability that a droplet will be produced having two or more receptor species in it. Thus, for example, at least about 50%, at least about 60%, at least about 70%, at least about 80%, at least about 90%, at least about 95%, at least about 97%, at least about 98%, or at least about 99% of the droplets may contain either no receptor species or only one receptor species.

The at least some microreactors may further comprise, in context of the present invention, a reverse transcriptase and barcoded primers, as further defined herein below.

### Recognition Assay and Classification

By “recognition” is meant herein a binding between a ligand species and a receptor species, preferably inducing a particular reaction by the ligand species and/or the receptor species.

As will be understood by the skilled person, the reaction induced by a recognition between a ligand species and a receptor species will depend on the particular ligands and receptors considered. For example, the recognition between a T cell antigen and its corresponding TCR displayed by a T cell will induce the activation of said T cell. Similarly, the recognition between a B cell antigen and its corresponding B cell receptor displayed by a B cell will induce the activation of said B cell.

Accordingly, the assay used to determine the recognition between a ligand species and a receptor species will depend on the particular ligands and receptors considered.

In a particular embodiment, when the receptor species of the set of receptors are displayed by cells, the recognition between ligands and receptors in each microreactor is assayed by determining if a cellular response is induced in said microreactor, the microreactor being classified as positive (recognition between a ligand species and a receptor species in the microreactor) when an induced cellular response is determined in said microreactor, and the microreactor being classified as negative (no recognition between a ligand species and a receptor species in the microreactor) when no induced cellular response is determined in said microreactor.

More particularly, when the set of receptors is TCR displaying T cells and the set of ligands is T cell antigen-displaying APCs, the recognition between ligands and receptors is assayed by determining if T cell activation is induced in said microreactor, the microreactor being classified as positive (recognition between a ligand species and a receptor species in the microreactor) when an induced T cell activation is determined in said microreactor, and the microreactor being classified as negative (no recognition between a ligand species and a receptor species in the microreactor) when no induced T cell activation is determined in said microreactor.

By “T cell activation” is meant herein the regulated series of events induced by the recognition of antigen whatever its chemical nature with a TCR which results in activation, differentiation, proliferation and acquisition of the T cell immunologic function by the TCR-displaying T cell. As well-known from the skilled person, signaling cascades initiated by TCR activation include the inositol tri-phosphate/Ca²⁺, diacylglycerol/protein kinase C, Ras/mitogen-activated protein kinase, and the PI 3-K pathways. Components of these pathways transmit information into the nucleus to activate the genes that code for a variety of secreted factors, such as IL-2, IL-4, IL-7, IL-9, IL-10, TNF-α, and interferon-γ, to activate the genes that code for a variety of cell surface expressed activation markers, such as CD137, CD40L and CD69, and to induce Caspase 3/7 pathway, associated with the proliferation, maturation, and function of cellular components of the immune system.

In certain embodiments, contacting the set of ligand species with the set of receptor species in the microreactor occurs for a short incubation period, e.g., about 0.001 hour to about 8 hours. In certain embodiments, the contacting step occurs for about 0.001 hour to about 8 hours, about 0.001 hour to about 7 hours, about 0.001 hour to about 6 hours, about 0.001 hour to about 5 hours, about 0.001 hour to about 4 hours, about 0.001 hour to about 3 hours, about 0.001 hour to about 2 hours, about 0.001 hour to about 1 hour, about 0.01 hours to about 8 hours, about 0.01 hour to about 7 hours, about 0.01 hour to about 6 hours, about 0.01 hour to about 5 hours, about 0.01 hour to about 4 hours, about 0.01 hour to about 3 hours, about 0.01 hour to about 2 hours, about 0.01 hour to about 1 hour, about 0.1 hour to about 8 hours, about 0.1 hour to about 7 hours, about 0.1 hour to about 6 hours, about 0.1 hour to about 5 hours, about 0.1 hour to about 4 hours, about 0.1 hour to about 3 hours, about 0.1 hour to about 2 hours, about 0.1 hour to about 1 hour, about 1 hour to about 8 hours, about 1 hour to about 7 hours, about 1 hour to about 6 hours, about 1 hour to about 5 hours, about 1 hour to about 4 hours, about 1 hour to about 3 hours, or about 1 hour to about 2 hours.

In certain embodiments, contacting the set of ligand species with the set of receptor species in a microreactor occurs for a long incubation period, e.g., at least about 8 hours, preferably about 8 hours to about 48 hours. In certain embodiments, the contacting step occurs for about 8 hours to about 48 hours, about 8 hours to about 40 hours, about 8 hours to about 32 hours, about 8 hours to about 24 hours, about 8 hours to about 16 hours, about 16 hours to about 48 hours, about 16 hours to about 40 hours, about 16 hours to about 32 hours, about 16 hours to about 24 hours, about 24 hours to about 48 hours, about 24 hours to about 40 hours, about 24 hours to about 32 hours, about 32 hours to about 48 hours, about 32 hours to about 40 hours, or about 40 hours to about 48 hours.

In certain embodiments, when the contacting step is a shorter incubation period (e.g., about 0.001 hour to about 8 hours), and the ligand species and the receptor species bind with high affinity, the enhanced signal produced is an early marker or a late marker for T cell activation. If the ligand species and receptor species have a high affinity for each other, selective or specific binding can occur immediately, which can result in the immediate activation of the T cells (e.g., when the ligand species is a T cell antigen and the receptor species is a T cell receptor). Immediate activation of the T cells can result in the immediate expression of early markers, which can include, but are not limited to, CD69, CD107a, and a transferrin receptor. Immediate activation of the T cells can also result in the immediate expression of continually expressed markers, which can include, but are not limited to, IFNγ, CD25, CD154, TNF, IL-10, IL-2, and IL-lb. Immediate activation of the T cells can also result in the expression of late markers during the shorter incubation time, which can include, but are not limited to CD137, HLA-DR, VLA1, PTA1, CD71, CD27, PD-1, TIM3, LAG3, or CTLA4.

In certain embodiments, when the contacting step is a longer incubation time (e.g., for at least about 8 hours, preferably about 8 to about 48 hours), and the ligand species and the receptor species bind with high affinity, the enhanced signal produced can be an early marker or a late marker for T cell activation. As the incubation time is longer, immediate activation of the T cells can result in the production of either early or late markers, as indicated above. Additionally, as the incubation time is longer, the continually expressed markers can also be produced.

In certain embodiments, when the contacting step is a longer incubation time (e.g., for at least about 8 hours, preferably about 8 to about 48 hours), and the ligand species and the receptor species bind with low affinity, the enhanced signal produced is an early marker for T cell activation. With a lower affinity, it can take a longer incubation time to activate the T cells, and, thus, depending upon the incubation time, either early or later markers for T cell activation can be observed. By extending the contacting step for the ligand species and the receptor species with low affinity, the enhanced signal produced can eventually be a late marker for T cell activation.

In certain embodiments, the early marker for T cell activation can be, but is not limited to, CD69, CD107a, or a transferrin receptor.

In certain embodiments, the a continually expressed marker for T cell activation can be, but is not limited to, IFNγ, CD25, CD154, TNF, IL-10, IL-2, and IL-lb.

In certain embodiments, the late marker for T cell activation can be, but is not limited to, CD137, HLA-DR, VLA1, PTA1, CD71, CD27, PD-1, TIM3, LAG3, or CTLA4.

In certain embodiments, the signal is detected with an anti-CD69 antibody, an anti-CD107a antibody, an anti-transferrin receptor antibody, anti-CD137 antibody, an anti-HLA-DR antibody, an anti-VLA1 antibody, an anti-PTA1 antibody, an anti-CD71 antibody, an anti-CD27 antibody, an anti-PD1 antibody, an anti-TIM3 antibody, an anti-LAG3 antibody, or an anti CTLA4 antibody.

Assays to determine T cell activation are well-known from the skilled person and include among others, detection of an upregulation of CD69, CD137, CD134 (OX40) or CD40L, detection of cytokine secretion, detection of the induction of Caspase 3/7 pathway, as well as the secretion of perforin or granzyme, or TNF-α, and interferon-γ.

Selective and/or specific binding of a ligand species to a receptor species can be detected in fluorescent assays using fluorescent antibodies in the range of about 1 nM to about 100 nM, about 1 nM to about 75 nM, about 1 nM to about 50 nM, about 1 nM to about 25 nM, about 10 nM to about 100 nM, about 10 nM to about 75 nM, about 10 nM to about 50 nM, about 10 nM to about 25 nM, about 20 nM to about 100 nM, about 20 nM to about 75 nM, about 20 nM to about 50 nM, about 30 nM to about 100 nM, about 30 nM to about 75 nM, about 30 nM to about 50 nM, about 40 nM to about 100 nM, about 40 nM to about 75 nM, about 40 nM to about 50 nM, about 50 nM to about 100 nM, about 50 nM to about 75 nM, about 60 nM to about 100 nM, about 60 nM to about 75 nM, about 70 nM to about 100 nM, about 80 nM to about 100 nM, or about 90 nM to about 100 nM. Selective binding of a ligand species to a receptor species can be at about 1 nM, about 5 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, or about 100 nM.

T cell activation can also be detected by sequencing of T cell mRNA using barcoded cDNA primers to detect mRNAs expression patterns characteristic specific of activated T cells.

These cDNA primers typically comprise the same barcode sequence as the ligand-specific and receptor-specific cDNA barcoded primers in the same microreactor and thus the cDNAs comprise the same barcode sequences as comprised in the ligand and receptor cDNAs, allowing the ligands and receptors in positive droplets (which contain activated T cells) to be identified by sequencing. The cDNA primers may, optionally, also comprise Unique Molecular Identifiers (UMIs) (Kivioja et al. (2012). Nat. Methods, 9, 72-74; Islam et al. (2014). Nat. Methods, 11, 163-166), to facilitate quantification and normalization of mRNA expression. The number of reads, or, optionally, the number of UMIs, is used to quantify and normalize the mRNA expression.

Similarly, when the set of receptors is B cell receptor displaying B cells and the set of ligands is B cell antigens, the recognition between ligands and receptors is assayed by determining if B cell activation is induced in said microreactor, the microreactor being classified as positive (recognition between a ligand species and a receptor species in the microreactor) when an induced B cell activation is determined in said microreactor, and the microreactor being classified as negative (no recognition between a ligand species and a receptor species in the microreactor) when no induced B cell activation is determined in said microreactor.

By “B cell activation” is meant herein a process or activity that causes B cells to exhibit a phenotype of an activated B cell, and “activated B cell” describes B cells that can exhibit some of the following phenotypes: B cell activation can be measured by any methods known in the art to identify antibody production (induction of >50 fold increase of expression, up to 300 times) and surface expression versus secretion, antigen specificity, expression of CD138/CD38, high reticulum endoplasmic reticulation or abundance, antigen-mediated activation, T cell dependent activation, T cell-independent activation, (see Blood. 2003; 102:592-600, Blood. 2002; 99:2905-2912, and Blood. 2010; 116(18):3445-3455), etc.

Assays to determine B cell activation are well-known from the skilled person.

B cell activation can also be detected by sequencing of B cell mRNA using barcoded cDNA primers to detect mRNA expression patterns characteristic specific of activated B cells. These cDNA primers typically comprise the same barcode sequence as the ligand-specific and receptor-specific barcoded cDNA primers in the same microreactor and thus the cDNAs comprise the same barcode sequences as comprised in the ligand and receptor cDNAs, allowing the ligands and receptors in positive droplets (which contain activated B cells) to be identified by sequencing. The cDNA primers may, optionally, also comprise Unique Molecular Identifiers (UMIs), to facilitate quantification of mRNA expression. The number of reads, or, optionally, the number of UMIs, is used to quantify mRNA expression.

In a particular embodiment, assay reagents are added to the microreactors. Preferably, said assay reagents are co-compartmentalized with said ligand species and said receptor species during the co-compartmentalization step.

For example, when the microreactors are microfluidic droplets, said assay reagents can be included in the first fluid and/or the second fluid used for the formation of said droplets. Alternatively, said assay reagents may be provided through a third fluid.

Reagents can also be added to pre-formed droplets by a variety of methods known to the skilled person, including passive droplet coalescence (see Mazutis et al. (2009). Lab Chip, 9 (18), 2665-2672; Mazutis & Griffiths (2012) Lab Chip, 12:1800-1806), droplet coalescence driven by local heating from a focused laser (Baroud et al. (2007). Lab Chip 7:1029-1033) or using electric forces (Chabert et al. (2005) Electrophoresis, 26:3706-3715; Ahn et al. (2006) Appl. Phys. Lett., 88:264105; Link et al. (2006) Angew. Chem., Int. Ed., 45:2556-2560; Priest et al. (2006) Appl. Phys. Lett., 89:134101), or by injection of liquids into pre-formed droplets, for example using electrical forces (picoinjection) (Abate et al. (2010) Proc. Nat. Acad. Sci. USA, 107:19163-19166).

As will be understood by the skilled person, said assay reagents will depend on the particular recognition assay carried out.

In a particular embodiment, said assay reagents include a reporter reagent enabling the direct sorting of positive microreactors by detection techniques, as defined below.

Based on the results of the assay, which is performed in each microreactor, each microreactor can be classified as positive or negative.

In a particular embodiment, positive microreactors can be separated from negative microreactors, thereby forming a group of positive microreactors.

Said separation can be carried out by any technique well-known from the skilled person, which will depend on the type of microreactors used. In particular, said separation may be carried out by sorting of the microreactors, in particular of the microfluidic droplets, for example, by detecting a reporter reagent. Said separation may also be carried out by sorting of the microreactors by flow cytometry.

In a preferred embodiment, when the microreactors are droplets, the droplets will be sorted in a microfluidic device by dielectrophoresis (Ahn et al. (2006) Appl. Phys. Lett. 88:024104) or using surface acoustic waves (Franke et al. (2009) Lab Chip 9:2625-2627), triggered, for example, by detecting a fluorescent signal in the droplets (Baret et al. (2009) Lab Chip, 9:1850-1858) or using magnetophoretic forces or using pneumatic controllers (see Xi et al. (2017) Lab Chip 17:751-771).

Alternatively, based on the classification of the microreactors as positive or negative, a subset of positive microreactors can be only intellectually established, without physically forming a group of positive microreactors.

### Optional Additional Reagents and Treatments of Microreactors

In a particular embodiment, said microreactors, in particular, said positive microreactors, include additional reagents.

Additional reagents typically include a reverse transcriptase (RT), a cell lysis buffer, deoxynucleotide triphosphates (dNTPs) and a plurality of barcoded primers specific for a nucleic acid sequence encoding the ligand or ligand candidate and of barcoded primers specific for a nucleic acid sequence encoding the receptor, as defined below.

When the ligand is a tagged ligand, as defined above, the barcoded primers specific for a nucleic acid sequence encoding the ligand may be barcoded primers specific for a nucleic acid sequence encoding the tag of said ligand. Similarly, when the receptor is a tagged receptor, as defined above, the barcoded primers specific for a nucleic acid sequence encoding the receptor may be barcoded primers specific for a nucleic acid sequence encoding the tag of said receptor.

Accordingly, in a particular embodiment, additional reagents are added to the microreactors, in particular to the positive microreactors, said additional reagents comprising at least a reverse transcriptase (RT), deoxynucleotide triphosphates (dNTPs), and a plurality of barcoded primers specific for a nucleic acid sequence encoding the ligand (or the ligand's tag) and of barcoded primers specific for a nucleic acid sequence encoding the receptor (or the receptor's tag) and optionally a cell lysis buffer, wherein the barcoded primers specific for the ligand (or the ligand's tag)-encoding nucleic acid sequence comprise a primer sequence specific for the ligand (or the ligand's tag)-encoding nucleic acid sequence and a barcode sequence or barcode set of sequences, wherein the barcoded primers specific for the receptor (or the receptor's tag)-encoding nucleic acid sequence comprise a primer sequence specific for the receptor (or the receptor's tag)-encoding nucleic acid sequence and a barcode sequence or barcode set of sequences, and wherein the barcode sequence or barcode set of sequences contained in a microreactor is distinguishable from the barcode sequence or barcode set of sequences contained in other microreactors, but the barcoded primers specific for the ligand (or the ligand's tag)-encoding nucleic acid sequence and for the receptor (or the receptor's tag)-encoding nucleic acid sequence contained in a given microreactor carry a common barcode sequence or barcode set of sequences.

Accordingly, in a particular embodiment, additional reagents are added to the microreactors, in particular to the positive microreactors, said additional reagents comprising at least a reverse transcriptase (RT), deoxynucleotide triphosphates (dNTPs), and a plurality of primers, being optionally barcoded, that will serve as template for template switch reaction (as described in U.S. Pat. No. 5,962,272), where either free floating specific primers for a nucleic acid sequence encoding the ligand (or the ligand's tag) and of barcoded primers specific for a nucleic acid sequence encoding the receptor (or the receptor's tag) would be added and/or a poly dT primer and/or random primer would be optionally added and optionally a cell lysis buffer, wherein the primers, being optionally barcoded, specific for the template switching reaction comprise a primer sequence known for people skilled in the art to associate with non-templated nucleotides generated during the RT by the reverse transcriptase at the 3′ end of the cDNA, typically triple Cytosine (see Zajac et al. (2013) PLoS ONE 8:e85270), wherein the primers, being optionally barcoded, specific for the non-templated nucleotide (typically three cytosines) comprise a primer sequence specific for non-templated nucleotide and a barcode sequence or barcode set of sequences, and wherein the barcode sequence or barcode set of sequences contained in a microreactor is distinguishable from the barcode sequence or barcode set of sequences contained in other microreactors, but the barcoded primers specific for the non-templated nucleotides contained in a given microreactor carry a common barcode sequence or barcode set of sequences.

When the receptor is a TCR, the nucleic acid sequence encoding the receptor is preferably a nucleic acid sequence encoding an alpha T cell receptor, a beta T cell receptor, a gamma T cell receptor or a delta T cell receptor.

When the receptor is an antibody displaying B cell, the nucleic acid encoding the receptor is preferably a nucleic acid sequence encoding the antibody heavy chain variable domain or the antibody light chain variable domain.

When the receptor, is constituted of several polypeptides, each encoded by a separate gene, several distinct barcoded primers each specific for the nucleic acid sequence encoding one of said polypeptides are preferably added. In other words, when the receptor is constituted of a number n of polypeptides, each encoded by a separate gene, a first barcoded primer comprising a primer sequence specific for the nucleic acid sequence encoding a first polypeptide of said receptor is added, a second barcoded primer comprising a primer sequence specific for the nucleic acid sequence encoding a second polypeptide of said receptor is added, . . . and a n^{th} barcoded primer comprising a primer sequence specific for the nucleic acid encoding the n^{th} polypeptide of said receptor is added.

Similarly, when the ligand is constituted of several polypeptides, each encoded by a separate gene, several distinct barcoded primers each specific for the nucleic acid sequence encoding one of said polypeptides are preferably added. In other words, when the ligand is constituted of a number n of polypeptides, each encoded by a separate gene, a first barcoded primer comprising a primer sequence specific for the nucleic acid sequence encoding a first polypeptide of said ligand is added, a second barcoded primer comprising a primer sequence specific for the nucleic acid sequence encoding a second polypeptide of said ligand is added, . . . and a n^{th} barcoded primer comprising a primer sequence specific for the nucleic acid encoding the n^{th} polypeptide of said ligand is added.

In a particular embodiment, typically when the receptor is constituted of two polypeptides, in particular of two chains, encoded by two separate genes, the barcoded primers specific for a nucleic acid sequence encoding the receptor are two different barcoded primers, each specific for a nucleic acid sequence encoding one of the two polypeptides, in particular chains, constituting the receptor.

Typically, when the receptor is a TCR, the barcoded primers specific for a nucleic acid sequence encoding the TCR are two different barcoded primers, wherein one barcoded primer is specific for a nucleic acid sequence encoding the alpha T-cell receptor and a second barcoded primer is specific for a nucleic acid sequence encoding the beta T-cell receptor.

Alternatively, when the receptor is a TCR, the barcoded primers specific for a nucleic acid sequence encoding the TCR are two different barcoded primers, wherein one barcoded primer is specific for a nucleic acid sequence encoding the gamma T-cell receptor and a second barcoded primer is specific for a nucleic acid sequence encoding the delta T-cell receptor.

Typically, when the receptor is a BCR and/or an antibody (as defined above), the barcoded primers specific for a nucleic acid sequence encoding the BCR and/or the antibody (as defined above) are two different barcoded primers, wherein one barcoded primer is specific for a nucleic acid sequence encoding the λ or κ chain of the BCR and/or the antibody and a second barcoded primer is specific for a nucleic acid sequence encoding the γ, δ, ε, α, or μ chain of the BCR and/or the antibody.

In the context of the invention, “a nucleic acid sequence encoding” the ligand (or the ligand's tag) or the receptor (or the receptor's tag) can be any type of nucleic acid as defined in the section “Definition” above. It can in particular be a DNA molecule or an RNA molecule. In a particular embodiment, said nucleic acid sequence encoding the ligand (or the ligand's tag) or the receptor (or the receptor's tag) consists or comprises of the mRNA sequence encoding said ligand (or said ligand's tag) or said receptor (or said receptor's tag), of a fragment thereof or a complementary sequence thereof. In another particular embodiment, said nucleic acid sequence encoding the ligand (or the ligand's tag) or the receptor (or the receptor's tag) consists or comprises of the cDNA sequence encoding said ligand (or said ligand's tag) or said receptor (or said receptor's tag), or of a fragment thereof. In still another embodiment, said nucleic acid sequence encoding the ligand (or the ligand's tag) or the receptor (or the receptor's tag) consists or comprised of the gene encoding said ligand (or said ligand's tag) or said receptor (or said receptor's tag), or of a fragment thereof.

As defined above, the barcode sequence or barcode set of sequences contained in a microreactor is distinguishable from the barcode sequence or barcode set of sequences contained in other microreactors, but the barcoded primers specific for the ligand (or ligand's tag)-encoding nucleic acid sequence and for the receptor (or receptor's tag)-encoding nucleic acid sequence contained in a given microreactor carry a common barcode sequence or barcode set of sequences. In other words, each microreactor comprises a unique type of barcode sequence or barcode set of sequences, optionally comprised in several barcoded primers, preferably in association with different primer sequences, while a particular barcode sequence or barcode set of sequences is preferably never included in two different microreactors.

In some embodiments, said barcoded primers are delivered on particles. In particular, in a preferred embodiment, said barcoded primers are initially bound to a particle. Indeed, binding said barcoded primers initially to a particle facilitates the delivery of only one type of barcoded primer into each microreactor.

As used herein, the terms “particle” and “bead” are used interchangeably.

The “particle” in context of the present invention refers to a microparticle.

In one embodiment the particle is a hydrogel particle, a polymeric particle or a magnetic particle.

The particle may have irregular or regular shape. For example, the particle can be spherical, ellipsoidal, or cubic.

“Hydrogel particles” are for example described in the International Patent Application No. WO 2008/109176, entitled “Assay and other reactions involving droplets.” Examples of hydrogels include, but are not limited to agarose, poly(ethylene glycol) diacrylate, or acrylamide-based gels, such as bis-acrylamide, polyacrylamide, streptavidine acrylamide, poly-N-isopropylacrylamide, or poly N-isopropylpolyacrylamide or mixtures thereof. In one example the hydrogel particle comprises acrylamide, bis-acrylamide and strepatvidine acrylamide.

For example, an aqueous solution of a monomer may be dispersed in a microreactor, for instance a droplet, and then polymerized, e.g., to form a gel. Another example is a hydrogel, such as alginic acid that can be gelled by the addition of calcium ions. In some cases, gelation initiators (ammonium persulfate and TEMED for acrylamide, or Ca²⁺ for alginate) can be added to a microreactor, for instance a droplet, for example, by co-flow with the aqueous phase, by diffusion and/or co-flow through the oil phase, or by coalescence of two different drops, e.g., as discussed in U.S. patent application Ser. No. 11/360,845, filed Feb. 23, 2006, entitled “Electronic Control of Fluidic Species,” by Link, et ah, published as U.S. Patent Application Publication No. 2007/000342 on Jan. 4, 2007; or in U.S. patent application Ser. No. 11/698,298, filed Jan. 24, 2007, entitled “Fluidic Droplet Coalescence,” by Ahn, et al.

In another set of embodiments, the particles may comprise one or more polymers and are thus herein referred to as “polymeric particle.” Exemplary polymers include, but are not limited to, polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers.

In addition, in some embodiments, the particles may be magnetic and are thus referred to as “magnetic particle,” which could allow for the magnetic manipulation of the particles. For example, the particles may comprise iron or other magnetic materials. The particles could also be functionalized so that they could have other molecules attached, such as proteins, nucleic acids or small molecules.

In some embodiments, the particle may be fluorescent.

In some embodiments, the particle may be functionalized, in particular to facilitate its identification and/or its sorting, for example using histidine, Flag, HA, streptavidin, acrydite DNA or biotin.

In one embodiment, the particle comprises streptavidin. Streptavidin may be coupled to the surface of the particle defined herein above or be inside said particle.

In one embodiment the hydrogel particles have a size from 1 pL to 1000 pL, such as 1 pL to 500 pL, 1 pL to 400 pL, 1 pL to 400 pL, 1 pL to 300 pL, for example 5 pL to 300 pL, 5 pL to 250 pL, 5 pL to 200 pL, 10 pL to 250 pL, 10 pL to 200 pL, 20 pL to 150 pL, 30 pL to 100 pL, 40 pL to 90 pL, 50 pL to 60 pL preferably 60 pL to 100 pL.

It will be understood by the skilled in the art that binding the barcoded primers temporally to a particle permits to provide particles having a high amount of barcoded primers. Furthermore, binding the barcoded primers initially to the particle facilitates the introduction of barcoded primers into each microreactor, in particular into each droplet, wherein the barcoded primers have the same barcode sequence.

Accordingly, in one embodiment, the barcoded primer is covalently bonded or non-covalently bonded to the particle.

“Non-covalently bonded” herein refers, for example, to a streptavidin-biotin bond. Other non-covalent bonds are known to the skilled in the art, such as avidin biotin bonds or his tag and nickel bonds.

“Covalently bonded” herein refers for example to an amino bond or an acrydite phosphoramidite bond.

“Streptavidin” generally refers to a 52.8 kDa protein purified from the bacterium Streptomyces avidinii. Streptavidin homo-tetramers have an extraordinarily high affinity for biotin with a dissociation constant (Kd) on the order of ≈10⁻¹⁴ mol/L, the binding of biotin to streptavidin is one of the strongest non-covalent interactions known in nature.

In a preferred embodiment, the non-covalent bond is a streptavidin-biotin bond.

Streptavidin-Biotin bonds are known to the skilled in the art. Accordingly, in one embodiment the particle as herein defined comprises streptavidin. Accordingly, in the same embodiment, the barcoded primers, as herein defined comprise biotin. In other words, the barcoded primers are functionalized with biotin.

Independent of the type of bond used to link the barcoded primers to the particle, the barcoded primers may further comprise at least one linker sequence.

Accordingly, in a further embodiment, the barcoded primer further comprises at least one linker sequence, said linker sequence being preferably comprised at the 5′ end. Accordingly, in one embodiment, the barcoded primer comprises from 5′ to 3′ a linker sequence, a barcode sequence, and a primer sequence.

In one embodiment, the “linker sequence” is a sequence with which the barcoded primer is optionally bonded to the particle.

“Optionally bonded” herein refers to the possibility that once the barcoded primers bonded to the particle are loaded into the microreactor or the plurality of microreactors, the barcoded primers might be released from the particle, so that the microreactor comprises the particle and the barcoded primers, said barcoded primers being separated from said particle.

Preferably, the linker sequence is a cleavable linker sequence, e.g., that can be cleaved upon application of a suitable stimulus, such as enzymatic and/or photocleavage.

“Cleavable linkers” are well known to the skilled in the art and are further described in Leriche et al. (2012) Bioorg. Med. Chem. 20:571-582. They may include, but are not limited to, TEV, trypsin, thrombin, cathepsin B, cathepsin D, cathepsin K, caspase lumatrix metalloproteinase sequences, phosphodiester, phospholipid, ester, galactose, dialkyl dialkoxysilane, cyanoethyl group, sulfone, ethylene glycolyl disuccinate, 2-N-acyl nitrobenzenesulfonamide, a-thiophenylester, unsaturated vinyl sulfide, sulfonamide after activation, malonaldehyde (MDA)-indole derivative, levulinoyl ester, hydrazone, acylhydrazone, alkyl thioester, disulfide bridges, azo compounds, 2-Nitrobenzyl derivatives, phenacyl ester, 8-quinolinyl benzenesulfonate, coumarin, phosphotriester, bis-arylhydrazone, bimane bi-thiopropionic acid derivative, paramethoxybenzyl derivative, tert-butylcarbamate analogue, dialkyl or diaryl dialkoxysilane, orthoester, acetal, aconityl, hydrazone, b-thiopropionate, phosphoramidate, imine, trityl, vinyl ether, polyketal, alkyl 2-(diphenylphosphino)benzoate derivatives, allyl ester, 8-hydroxyquinoline ester, picolinate ester, vicinal diols, and selenium compounds. Cleavage conditions and reagents include, but are not limited to, enzymes, nucleophilic/basic reagents, reducing agents, photo-irradiation, electrophilic/acidic reagents, organometallic and metal reagents, and oxidizing reagents.

In a preferred embodiment, the cleavable linker is a photocleavable moiety, for example a photolabile chemical group followed a chain of 1 to 30 carbon atoms, typically a chain of 6 to 10 carbon atoms.

In a further preferred embodiment, the cleavable linker is a double-stranded DNA molecule containing a target site for a specific restriction endonuclease.

In a particular embodiment, the barcoded primers bound to a particle are released from the particle in the microreactor, in particular, prior to or after lysing the cells, as disclosed below.

The release of at least some of the barcoded primers may further occur after lysing the cells and before reverse transcribing the released nucleic acids hybridized to said barcoded primers or after lysing the cells and after reverse transcribing the released nucleic acids hybridized to said barcoded primers.

The skilled in the art will understand that depending on the time point selected for releasing the barcoded primers, the term “at least some of the barcoded primers” might refer to, for example, at least some of the barcoded primers hybridized to the nucleic acids released by the cells or a DNA/RNA duplex.

In one embodiment, the at least some of the barcoded primers can be released using any means, such as enzymes, nucleophilic/basic reagents, reducing agents, photo-irradiation, electrophilic/acidic reagents, organometallic and metal reagents, and oxidizing reagents.

In one embodiment, the at least some of the barcoded primers can be released using enzymatic and/or photocleavage. For example, an endonuclease may be used to cleave a linker sequence or any other sequence to release the at least some of the barcoded primers from the particle.

In a further embodiment, releasing the barcoded primer refers to disrupting the bond, such as a streptavidin biotin. Methods to disrupt a streptavidin biotin bond are known to the skilled in the art and include enzymatic digestion of streptavidin and/or denaturation of streptavidin.

In one embodiment, the barcoded primer is released by enzymatic digestion of streptavidin.

Preferably, each particle carries a barcode sequence or barcode set of sequences distinguishable from barcode sequences or barcode sets of sequences carried by other beads. In other words, each particle carries a unique majority type of barcode sequence or barcode set of sequences, optionally comprised in several barcoded primers, preferably at least some being in association with different primer sequences, while two different particles preferably do not carry the same majority barcode sequence or barcode set of sequences.

In a preferred embodiment, each microreactor contains a single particle carrying barcoded primers or less than 10 particles, in particular, less than 9, 8, 7, 6, 5, 4, 3, or 2 particles carrying barcoded primers. In a particularly preferred embodiment, each microreactor carries a single particle carrying barcoded primers.

The “reverse transcriptase (RT)” in context of the present invention is an enzyme used to generate complementary DNA (cDNA) from an RNA template, in a process termed reverse transcription.

In one embodiment, the reverse transcriptase is selected from the group consisting of Superscriptase I, Superscriptase II, Superscriptase III, Superscriptase IV, Murine Leukemia RT, SmartScribe RT, Maxima H RT, or MultiScribe RT.

In one embodiment, the reverse transcriptase is at a concentration of 1 to 50 U/μL, preferably 5 to 25 U/μL, for example at 12.5 U/μL.

In the context of the present invention, the “cell lysis buffer” is a composition enabling cell lysis, preferably without disruption of the microreactors, in particular, of the droplets.

Preferably, the cell lysis buffer is compatible with RT activity and/or with reagents used for the recognition assay.

In one embodiment, the lysis buffer comprises enzymes selected from the group consisting of lysozyme, lysostaphin, zymolase, mutanolysin, glycanases, proteases, and mannose.

In one preferred embodiment, the lysis buffer comprises magnesium chloride, a detergent, a buffered solution and an RNase inhibitor.

In one embodiment, the magnesium chloride is used at a concentration of between 1 mM to 20 mM.

In one embodiment, the detergent is selected from the group consisting of Triton-X-100, NP-40, Nonidet P40, and Tween-20 and IGEPAL CA 630.

In one embodiment, the detergent is at a concentration of 0.1% to 10%.

Non-limiting examples of the buffered solution include Tris-HCl, Hepes-KOH, Pipes-NaOH, maleic acid, phosphoric acid, citric acid, malic acid, formic acid, lactic acid, succinic acid, acetic acid, pivalic (trimethylacetic) acid, pyridine, piperazine, picolinic acid, L-histidine, MES, Bis-tris, bis-tris propane, ADA, ACES, MOPSO, PIPES, imidazole, MOPS, BES, TES, HEPES, DIPSO, TAPSO, TEA (triethanolamine), N-Ethylmorpholine, POPSO, EPPS, HEPPS, HEPPSO, Tris, tricine, Glycylglycine, bicine, TAPS, morpholine, N-Methyldiethanolamine, AMPD (2-amino-2-methyl-1,3-propanediol), Diethanolamine, AMPSO, boric acid, CHES, glycine, CAPSO, ethanolamine, AMP (2-amino-2-methyl-1-propanol), piperazine, CAPS, 1,3-Diaminopropane, CABS, or piperidine (see also, www.reachdevices.com/Protein/Biological Buffers.html).

Non-limiting examples of RNase inhibitors include RNase OUT, IN, SuperIN Rnase, and those inhibitors targeting a wide range of RNAse (e.g., A, B, C, 1 and T1).

In one example the lysis buffer is typically 0.36% Igepal CA 630, 50 mM Tris-HCl pH 8.

In a particular embodiment, said additional reagents are added into the microreactor, in particular into the microfluidic droplet, by injection from a reservoir, for example using electrical forces (picoinjection) (Abate et al. (2010) Proc. Nat. Acad. Sci. USA 107:19163-19166).

In another particular embodiment, said additional reagents are added into the microreactor, in particular into the microfluidic droplet, by coalescence with a second microreactor, in particular a second microfluidic droplet, comprising said additional reagents but not comprising any ligand or receptor. Droplets can be coalesced by a variety of methods known to the skilled person, including passive droplet coalescence (see Mazutis et al. (2009) Lab on a Chip, 9(18):2665-2672; Mazutis et al. (2012) Lab Chip, 12:1800-1806), droplet coalescence driven by local heating from a focused laser (Baroud et al. (2007) Lab Chip 7:1029-1033) or using electric forces (Chabert et al. (2005) Electrophoresis 26:3706-3715; Ahn et al. (2006) Appl. Phys. Lett., 88:264105; Link et al. (2006) Angew. Chem., Int. Ed., 45:2556-2560; Priest et al. (2006) Appl. Phys. Lett. 89:134101) or using magnetophoretic forces or using pneumatic controllers (see Xi et al. (2017) Lab Chip 17:751-771).

Said second microreactor, in particular said second microfluidic droplet, can be prepared by the same techniques as those disclosed above for the microreactors comprising the ligands and receptors.

By “coalescence” is meant herein the process by which two or more droplets or particles merge during contact to form a single daughter droplet or particle.

Preferably, said additional reagents are selectively added to positive microreactors, in particular, after the separation step of the positive microreactors from the negative microreactors.

In a particular embodiment, in each microreactor, in particular in each positive microreactor, in which the above additional reagents are added, barcoded cDNAs are prepared by (a) lysing the cells expressing or displaying receptors and the cells expressing or displaying ligands, to release mRNA from the cells, (b) hybridizing at least some of the released mRNA coding for the receptor (or for the receptor's tag) to the receptor (or the receptor's tag)-encoding nucleic acid sequence specific primer, being optionally barcoded, and at least some of the released mRNA coding for the ligand (or for the ligand's tag) to the ligand (or the ligand's tag)-encoding nucleic acid sequence specific barcoded primer, in at least some of the microreactors, and (c) reverse transcribing the released mRNA hybridized to the primers, being optionally barcoded thereby obtaining barcoded cDNAs.

As will be understood by the skilled person, when the ligand's tag or the receptor's tag is a barcode sequence, it is not necessary to prepare barcoded cDNAs as detailed above, since the nt sequences of the ligand or the receptor allow themselves their own identification.

“Barcoding” herein refers to adding a genetic sequence, a so-called barcode sequence as further defined herein above, to a nucleic acid which allows to distinguish said barcoded nucleic acid from a nucleic acid having another added genetic sequence, i.e., another unique barcode sequence.

The term “cell lysis” in the context of the present invention may be accomplished by enzymatic, physical, and/or chemical means, or any combination thereof, in particular enzymatic, physical, and/or chemical means. Other cell disruption methods may also be used.

Accordingly, in one embodiment, the cells are lysed using enzymatic, physical, and/or chemical cell lysis.

“Enzymatic methods” to remove cell walls is well-established in the art. The enzymes are generally commercially available and, in most cases, were originally isolated from biological sources. Enzymes commonly used include lysozyme, lysostaphin, zymolase, mutanolysin, glycanases, proteases, and mannose.

As known by the skilled in the art “chemical cell lysis” is achieved using chemicals such as detergents, which disrupt the lipid barrier surrounding cells by disrupting lipid-lipid, lipid-protein, and protein-protein interactions. The ideal detergent for cell lysis depends on cell type and source. Nonionic and zwitterionic detergents are milder detergents. The Triton X series of nonionic detergents, the IGEPAL CA 630 nonionic detergent, and 3-[(3-Cholamidopropyl) dimethylammonio]-1-propanesulfonate (CHAPS), a zwitterionic detergent, are commonly used for these purposes. In contrast, ionic detergents are strong solubilizing agents and tend to denature proteins, thereby destroying protein activity and function. SDS, an ionic detergent that binds to and denatures proteins, is used extensively in the art to disrupt cells.

“Physical cell lysis” refers to the use of sonication, thermal shock (above 40° C., below 10° C.), electroporation, or laser-induced cavitation.

In one example the cells are lysed on ice.

In one preferred embodiment, the cell lysis does not disrupt or destroy the microreactors, in particular, the droplets, in the context of the invention.

The term “hybridization,” as described herein, refers to a phenomenon in which the primer sequence present in the barcoded primer anneals to a complementary nucleic acid sequence of the released nucleic acids. Accordingly, as known by the skilled in the art, the temperature to use depends on the primer sequence and/or the polymerase enzyme used.

The step of reverse transcription defined above refers to reverse transcribing the released nucleic acids hybridized to said barcoded primers using the primer sequence in at least some of the microreactors. Reverse transcription is performed using the reverse transcriptase (RT) comprised in at least some of the microreactors.

“Reverse Transcription” or “RT reaction” is a process in which single-stranded RNA is reverse transcribed into a single-stranded complementary DNA (cDNA) by using total cellular RNA or poly(A) RNA, a reverse transcriptase enzyme, a primer, dNTPs and an RNase inhibitor. It will be understood by the skilled in the art, that the product of the reverse transcription is a RNA/DNA duplex comprising a single strand cDNA hybridized to its template RNA. As it will be further understood, said RNA/DNA duplex is further linked to the barcoded primer comprising the primer sequence used for the reverse transcription.

“Template switching” refers to a technology described originally in 2001, frequently referred to as “SMART” (switching mechanism at the 5′ end of the RNA transcript) technology (Takara Bio USA, Inc). This technology has shown promise in generating full-length cDNA libraries, even from single-cell-derived RNA samples (Zhu et al. (2001) Biotechniques 30:892-897). This strategy relies on the intrinsic properties of Moloney murine leukemia virus (MMLV) reverse transcriptase and the use of a unique template switching oligonucleotide (TS oligo, or TSO). During first-strand synthesis, upon reaching the 5′ end of the RNA template, the terminal transferase activity of the MMLV reverse transcriptase adds a few additional nucleotides (mostly deoxycytidine) to the 3′ end of the newly synthesized cDNA strand. These bases function as a TS oligo-anchoring site. Upon base pairing between the TS oligo and the appended deoxycytidine stretch, the reverse transcriptase “switches” template strands, from cellular RNA to the TS oligo, and continues replication to the 5′ end of the TS oligo. By doing so, the resulting cDNA contains the complete 5′ end of the transcript, and universal sequences of choice are added to the reverse transcription product. Along with tagging of the cDNA 3′ end by oligo dT primers, this approach makes it possible to efficiently amplify the entire full-length transcript pool in a completely sequence-independent manner (Shapiro et al. (2013) Nat. Rev. Genet. 14:618-630).

Accordingly, it will be understood by the skilled in the art, that after reverse transcribing the nucleic acids, the microreactor further comprises cDNAs.

Accordingly, in one embodiment, at least some of the microreactors further comprise cDNAs produced by reverse transcription of nucleic acids from the cells contained in said microreactors.

In one embodiment, said cDNA refers to a single-stranded complementary DNA.

In a further embodiment, said cDNA is comprised in a RNA/DNA duplex.

In one embodiment, the RNA/DNA duplex refers to the RNA that has been reverse transcribed and is hybridized to the primer sequence of at least one of the primers, which is optionally barcoded, contained in the microreactor.

As it will be understood by the skilled in the art, in one embodiment, the RNA/DNA duplex is linked to the primer, which is optionally barcoded, comprising the primer sequence to which the nucleic acid, preferably mRNA, was hybridized and which was used for reverse transcription.

In one example, hybridization and reverse transcription are performed by incubating the microreactors for example for 1 h or 2 h at 55° C. or 50° C. during typically mixing of the microreactors at for example 550 rpm.

### Identification of Ligand Species and Receptor Species

Identification of the ligand species and the receptor species contained in each microreactor, in particular, each positive microreactor, can be carried out by any technique well-known from the skilled person. In particular, identification of the ligand species and the receptor species contained in each microreactor, in particular each positive microreactor, can be carried out by sequencing, in particular, by sequencing DNA, the barcoded cDNAs obtained as detailed above, or the tags.

In one embodiment, the barcoded cDNAs produced by the reverse transcription as defined above are recovered and further used for identification, typically, by subsequent amplification and sequencing library preparation.

Accordingly, in one embodiment, the method of the invention further comprises recovering cell cDNAs produced by reverse transcription in at least some of the microreactors, preferably in the positive microreactors.

“Recovering” herein refers to isolating the barcoded cDNAs produced by reverse transcription in at least some of the microreactors from said plurality of microreactors.

In one embodiment, recovering herein refers to collecting the microreactors comprising barcoded cDNA produced by reverse transcription or collecting the aqueous composition contained in said microreactors comprising said barcoded cDNA, and separating the barcoded cDNA comprised in the aqueous composition.

In one particular embodiment, recovering herein refers to collecting the microfluidic droplets comprising barcoded cDNA produced by reverse transcription, breaking the microfluidic droplets and separating the barcoded cDNA comprised in the aqueous composition from the oil phase of said microfluidic droplets.

Methods to isolate nucleic acids, in particular cDNA from microfluidic droplets are known to the skilled in the art and comprise for example, collecting the microfluidic droplets and breaking the emulsion by, for example, applying an electrical field (electrocoalescence) or by adding a chemical emulsion breaking agent, such as perfluoro-octanol in the case of droplets in fluorinated carrier oils. In one example, the broken emulsion is typically centrifuged for, for example, 10 minutes at 10,000 g at 4° C. and the supernatant comprising the barcoded cDNA in the aqueous phase is recovered.

In one embodiment, the method further comprises the step of removing unincorporated barcoded primers from the aqueous composition of the microreactors. In one preferred embodiment, the step of removing unincorporated barcoded primers from the aqueous composition of at least some of the microreactors takes place after the step of recovering the barcoded cDNA produced by reverse transcription as defined herein above.

Preferably, the step of removing unincorporated barcoded primers precedes the amplification step and/or the sequencing step defined herein below.

In one embodiment, removing unincorporated barcoded primers comprises contacting the aqueous composition of the at least some of the microreactors with a purification substrate wherein the purification substrate removes unincorporated barcoded primers. In one embodiment, the purification substrate comprises beads or particles, which, optionally, form a column. In a further example, unincorporated barcoded primers are removed by size selection using for example an acrylamide or an agarose gel.

In one embodiment, the step of removing unincorporated barcoded primers comprises contacting the aqueous composition of the at least some of the microreactors with an exonuclease, such as the exonuclease Exo1, to degrade the unincorporated barcoded primers within the aqueous composition of the at least some of the microreactors.

In certain embodiments of this step, the exonuclease degrades single stranded nucleic acid sequences from the aqueous compositions comprising the cDNA.

It will be understood by the skilled in the art, that the barcoded cDNA obtained after reverse transcription is typically present in the form of a RNA/DNA complex and thus protected from said exonucleases.

In one embodiment, the barcoded cDNA comprises one or more modified nucleotides or nucleotide analogs, for example for facilitating purification of the barcoded cDNA sequences or molecules.

For example, the nucleotides may be employed as phosphorothioate derivatives (replacement of a non-bridging phosphoryl oxygen atom with a sulfur atom) which have increased resistance to nuclease digestion. 2′-methoxyethyl (MOE) modification (such as the modified backbone commercialized by ISIS Pharmaceuticals) is also effective.

Other examples of modified nucleotides include derivatives of nucleotides with substitutions at the 2′ position of the sugar, in particular with the following chemical modifications: O-methyl group (2′-O-Me) substitution, 2-methoxyethyl group (2′-O-MOE) substitution, fluoro group (2′-fluoro) substitution, chloro group (2′-Cl) substitution, bromo group (2′-Br) substitution, cyanide group (2′-CN) substitution, trifluoromethyl group (2′-CF₃) substitution, OCF₃ group (2′-OCF₃) substitution, OCN group (2′-OCN) substitution, O-alkyl group (2′-O-alkyl) substitution, S-alkyl group (2′-S-alkyl) substitution, N-alkyl group (2′-N-akyl) substitution, O-alkenyl group (2′-O-alkenyl) substitution, S-alkenyl group (2′-S-alkenyl) substitution, N-alkenyl group (2′-N-alkenyl) substitution, SOCH₃ group (2′-SOCH₃) substitution, SO₂CH₃ group (2′-SO₂CH₃) substitution, ONO₂ group (2′-ONO₂) substitution, NO₂ group (2′-NO₂) substitution, N₃ group (2′-N₃) substitution and/or NH₂ group (2′-NH₂) substitution. Other examples of modified nucleotides include biotin labeled nucleotides.

Other examples of modified nucleotides include nucleotides wherein the ribose moiety is used to produce locked nucleic acid (LNA), in which a covalent bridge is formed between the 2′ oxygen and the 4′ carbon of the ribose, fixing it in the 3′-endo configuration.

Other examples of nucleotide analogs include deoxyinosine.

Other examples of nucleotide analogs include Biotinylated, fluorescently labelled nucleotide. For example, Biotin-11-dCTP can be used as a substrate for the reverse transcriptase to incorporate biotins into the cDNA during polymerization, allowing affinity purification using streptavidin or avidin.

In one embodiment, the barcoded cDNA is further treated with RNAse A and/or RNAse H.

“RNAse A” is an endoribonuclease that specifically degrades single-stranded RNA at C and U residues. In one embodiment, the RNAse A is at a concentration of 10 to 1000 μg/μL, preferably 50 to 200 μg/μL, for example at 100 μg/μL.

“RNAse H” is a family of non-specific endonucleases that catalyze the cleavage of RNA via a hydrolytic mechanism. RNase H ribonuclease activity cleaves the 3′-O—P bond of RNA in a DNA/RNA duplex substrate to produce 3′-hydroxyl and 5′-phosphate terminated products. In one embodiment, the RNAse H is at a concentration of 10 to 1000 μg/μL, preferably 50 to 200 μg/μL, for example at 100 μg/μL.

In one embodiment, the barcoded cDNA is further treated with Proteinase K. “Proteinase K” is a broad-spectrum serine protease and digests proteins, preferentially after hydrophobic amino acids. In one embodiment, the Proteinase K is at a concentration of 0.1 to 5 mg/mL, preferably 0.1 to 1 mg/mL, for example at 0.8 mg/mL.

In one embodiment, the barcoded cDNAs obtained after reverse transcription are sequenced to allow identification of receptors and ligands contained in the same microreactor.

In one embodiment, the step of sequencing the barcoded cDNA may comprise performing a next generation sequencing (NGS) protocol on a sequencing library. Any type of NGS protocol can be used such as the MiSeq Systems (Illumina®), the HiSeq Systems (Illumina®), the NextSeq System (Illumina®), the NovaSeq Systems (Illumina®), the IonTorrent system (ThermoFisher), the IonProton system (ThermoFisher), or the sequencing systems produced by Pacific Biosciences or by Nanopore.

In certain embodiments, the NGS protocol comprises loading an amount of the sequencing library between 1 pM and 20 pM, in particular between 1.5 pM and 20 pM, per flow cell of a reagent kit.

In one embodiment, the NGS sequencing protocol further comprises the step of adding 5-60% PhiX to the amount of the sequencing library or to the flow cell of the reagent kit.

In one embodiment, prior to sequencing, the barcoded cDNAs are further amplified.

In one embodiment, the amplification step is performed by a polymerase chain reaction (PCR), and/or a linear amplification.

In one embodiment, the linear amplification precedes the PCR reaction.

In one embodiment, the linear amplification is an in vitro transcription.

In one embodiment, the linear amplification is an isothermal amplification.

In one embodiment, said amplification step is performed after removing unincorporated barcoded primers. In one embodiment, said amplification step is performed prior to the sequencing step defined herein above.

In one embodiment, the barcoded cDNA produced after reverse transcription is quantified using qPCR.

In one embodiment, specific sequences necessary for sequencing are added during amplification or by ligation of adaptors, thereby generating a sequencing library.

As will be understood by the skilled person, since the barcoded cDNAs from a particular microreactor carry a same specific majority barcode sequence or barcode set of sequences which is different from the majority barcode sequences or barcode sets of sequences included in other microreactors, it is possible to determine which identified ligand species were contained in the same microreactor, in particular in positive microreactors, as a particular identified ligand receptor.

### Embodiments

The invention also provides the following non-limiting embodiments.

Embodiment 1 is a method of identifying a cognate pair of a ligand species and a receptor species, the method comprising:
a. providing a set of ligand species, wherein each ligand species is represented at least one time;
b. providing a set of receptor species, wherein each receptor species is represented at least one time;
c. contacting the set of ligand species with the set of receptor species in a microreactor, wherein upon selective binding of a ligand species with a receptor species an enhanced signal is produced;
d. detecting a cognate pair of ligand species and receptor species by the production of the signal; and
e. identifying the cognate pair of ligand species and receptor species.

Embodiment 2 is the method of embodiment 1, wherein each ligand species comprises a barcode sequence.

Embodiment 3 is the method of embodiment 1 or 2, wherein each receptor species comprises a barcode sequence.

Embodiment 4 is the method of any one of embodiments 1-3, wherein each ligand species is expressed by or displayed on the surface of a cell or bead or is expressed or present in a cell free extract or in solution.

Embodiment 5 is the method of embodiment 4, wherein the ligand species is expressed by or displayed on the surface of an antigen-presenting cell.

Embodiment 6 is the method of embodiment 5, wherein the antigen-presenting cell is selected from a macrophage, a dendritic cell, a Langerhans cell, a B cell, a monocyte derived dendritic cell, or another cell expressing a MHC class I or II molecule.

Embodiment 7 is the method of any one of embodiments 1-6, wherein each receptor species is expressed by or displayed on the surface of a cell or bead or is expressed or present in a cell free extract or in solution.

Embodiment 8 is the method of any one of embodiments 1-7, wherein the microreactor is selected from an aqueous droplet, a microcapsule, a microbead, a compartment of a microfluidic chip, or a well.

Embodiment 9 is the method of any one of embodiments 1-8, wherein the signal is selected from a morphological change of any one of a cell, a ligand, or a receptor; a fluorescent signal enhancement; a modification of a fluorescent signal using a caged compound or by a quenching reaction; a light absorption; a visible structure modification/creation; or a combination of signals thereof.

Embodiment 10 is the method of any one of embodiments 2-9, wherein identifying the cognate pair of ligand species and receptor species comprises amplifying the ligand species and/or the receptor species, wherein at least one of the amplified ligand species and receptor species are sequenced for identification.

Embodiment 11 is the method of any one of embodiments 1-10, wherein the set of ligand species is selected from T cell antigens, B cell antigens, viral antigens, bacterial antigens, parasitic antigens, neoantigens, tumor associated antigens (TAAs), tumor specific antigens, immune checkpoint molecules, cytokines, carbohydrates, members of the immunoglobulin superfamily, selectins, chemokines, hormone, growth factors, G-protein coupled receptor ligands, or enzyme substrates.

Embodiment 12 is the method of any one of embodiments 1-11, wherein the set of receptor species is selected from T cell receptors, B cell receptors, immune checkpoint receptors, cytokine receptors, selectins, integrins, members of the immunoglobulin superfamily, cadherins, chemokine receptors, hormone receptors, growth factor receptors, G-protein coupled receptors (GPCRs), or enzymes.

Embodiment 13 is the method of any one of embodiments 1-12, wherein the ligand species is a T cell antigen and the receptor species is a T cell receptor, and upon selective binding of the T cell antigen with the T cell receptor, the enhanced signal is produced, wherein the enhanced signal produced is the result of T cell activation.

Embodiment 14 is the method of any one of embodiments 1-12, wherein the ligand species is a viral antigen and the receptor species is a T cell receptor, and upon selective binding of the viral antigen with the T cell receptor, the enhanced signal is produced, wherein the enhanced signal produced is the result of T cell activation.

Embodiment 15 is the method of embodiment 13 or 14, wherein contacting the set of ligand species with the set of receptor species in a microreactor occurs for about 0.001 hour to about 8 hours.

Embodiment 16 is the method of embodiment 13 or 14, wherein contacting the set of ligand species with the set of receptor species in a microreactor occurs for at least about 8 hours.

Embodiment 17 is the method of embodiment 15 or 16, wherein the ligand species and the receptor species bind with high affinity and the enhanced signal produced is an early marker or late marker for T cell activation.

Embodiment 18 is the method of embodiment 16, wherein the ligand species and the receptor species bind with low affinity and the enhanced signal produced is an early marker or late marker for T cell activation.

Embodiment 19 is the method of embodiment 17 or 18, wherein the early marker for T cell activation is selected from CD69, CD107a, or a transferrin receptor.

Embodiment 20 is the method of embodiment 17 or 18, wherein the late marker for T cell activation is selected from CD137, HLA-DR, VLA1, PTA1, CD71, CD27, PD-1, TIM3, LAG3, or CTLA4.

Embodiment 21 is the method of embodiment 19 or 20, wherein the enhanced signal is detected with an anti-CD69 antibody, an anti-CD107a antibody, an anti-transferrin receptor antibody, anti-CD137 antibody, an anti-HLA-DR antibody, an anti-VLA1 antibody, an anti-PTA1 antibody, an anti-CD71 antibody, an anti-CD27 antibody, an anti-PD1 antibody, an anti-TIM3 antibody, an anti-LAG3 antibody, or an anti CTLA4 antibody.

Embodiment 22 is the method of any one of embodiments 1-12, wherein the ligand species is a B cell antigen and the receptor species is a B cell receptor, and upon selective binding of the B cell antigen with the B cell receptor, the enhanced signal is produced.

Embodiment 23 is the method of embodiment 22, wherein the signal is detected with an anti-CD138 antibody, an anti-CD19 antibody, an anti-CD45R antibody, an anti-CD45 antibody, an activation of fluorescent reporter expression, or an inhibition of fluorescent reporter expression.

### EXAMPLES

The following examples of the invention are to further illustrate the nature of the invention. It should be understood that the following examples do not limit the invention and that the scope of the invention is to be determined by the appended claims.

Materials and Methods

Cell preparation: T cells were cultured in X vivo with 5% human serum supplemented with 1% penicillin, streptomycin, 1% sodium pyruvate and non-essential amino acids (GIBCO). Cells were counted, washed with PBS, and spun down at 450 g, for 5 minutes at 4° C. Cell pellets were resuspended in PBS at 2 M/ml and stained with cell trace already dissolved in DMSO at 5 μM, except cell trace red at 1 μM, at 37° C. for 20 minutes. Buffer with protein (complete medium) was added at least 5 times more and incubated 5 minutes at room temperature to stop the reaction. Cells were there spun down at 450 g for 3 minutes at 4° C. and resuspended in MACS buffer (phosphate-buffered saline (PBS), pH 7.2, 0.5% bovine serum albumin (BSA), and 2 mM EDTA by diluting MACS® BSA Stock Solution (#130-091-376) 1:20 with autoMACS® Rinsing Solution (#130-091-222).

T cell purification: T cells were purified with negative selection as recommended by manufacturer (Miltenyi; Bergisch Gladbach, Germany). PBMC were resuspend in MACs buffer at 250 M/ml. Antibody cocktails targeting another subset was added at 1/5 dilution and incubated for 5 minutes at 4° C. Cell concentration was adjusted to 125 M/ml with MACs buffer and Microbead cocktail was added at 1/5 dilution. T cells were extracted using LS column in the magnetic field of MACS separator.

K562 loading with peptide: K562 cells were counted, washed with PBS, and spun down at 450 g for 5 minutes. The cell pellet was resuspended in PBS at 2 M/ml and stained with cell trace at the recommended concentrations. The staining reaction was stopped with X vivo 5% human serum for 5 minutes at room temperature. Cells were then washed and resuspended at 1 M/ml in x vivo with 5% human serum. Peptide was then added to the cell suspension at 10 μg/ml and incubated at 37° C., 5% CO₂ for 90 minutes to allow peptide presentation by HLA0201.

mRNA transfection: Media for K562 cells was replaced every 2-3 days. The K562 cells must be in growth phase and below passage 10 (passage cells after reaching 1 M:m1) for mRNA transfection. K562 cells were seeded at 1×10⁵ cells/ml, and the cells were cultured for 2 days before Nucleofection™ at a density of 3×10⁵ cells/ml. After uploading the experimental parameter file, the appropriate Nucleofector™ Program (FF-120) was selected. Cell culture plates were prepared by filling appropriate number of wells with desired volume of recommended culture media and pre-incubated/equilibrated in a humidified 37° C. 5% CO₂ incubator. K562 cells were counted, and the required cell number was centrifuged at 90 g for 10 minutes at room temperature. The supernatant was completely removed, and the cells were resuspended in 4D Nucleofector solution at the appropriate concentration (10 M/ml). 1 M of cells was mixed with the required amount of mRNA substrates (10 μg) and transferred into the Nucleocuvette™ vessels that have been placed with a closed lid into the retainer of the 4D-Nucleofector™ X Unit. After transfection with pulse, the Nucleocuvette™ Vessel was carefully removed from the retainer and incubated 10 minutes at room temperature. Cells were then resuspended in pre-warmed medium (500 μl per 1 M of cells), and the cells were mixed gently by pipetting up and down two to three times and plated. The plated cells were then incubated in a humidified 37° C. 5% CO₂ incubator until analysis.

Cell preparation for IFN-γ in droplet: For effector cells, T cells were labeled with cell Trace yellow, and then with a bispecific antibody targeting CD45 and IFNγ, at 1/10 dilution in cooled medium for 5 minutes at 4° C. and at a cell concentration 10 M/ml. T cells were then washed and resuspended in co-flow containing X vivo medium with 5% human serum supplemented with Pluronic F68 at 0.10%, 23.6% of Nycodenz, 6% DNA marker (Nucgreen).

For peptide loading, K562 cells were labeled with indicated cell trace at 5 μM, and then either not loaded or loaded with peptide at 10 μM. The cells were then washed by adding cold buffer, and the cells were spun down for 10 minutes at 4° C., and then resuspended in co-flow, as described above. For mRNA transfected K562 cells, cells were not labeled as transfected mRNA encode for a fluorescent reporter.

Detection antibody was added into the cell suspension. At the end, these cells were co-flowed separately into a microfluidic device, and, thus 100 picoliter droplets were produced, and then incubated overnight at 37° C. 5% CO₂.

### Droplet Production

Aqueous phase I: Preparation of cells for compartmentalization in droplets. Cell suspensions were prepared for compartmentalization in droplets at 4° C. as described, and cells were resuspended in X vivo supplemented with 0.1% Pluronic F68 (LifeTechnologies; Carlsbad, CA), 25 mM HEPES pH 7.4 (LifeTechnologies), 1% Pen/Strep (ThermoFisher; Waltham, MA), 23.6% of Nycodenz (Fisher Scientifique) and 6% DNA marker (Nucgreen-Thermofisher) so as to achieve a λ (mean number of cells per droplet) of ˜0.9 and 0.45 for K562 and T cells respectively, in T cell activation assay in droplet.

Aqueous phase II: Preparation of bioassay reagents and reporter cells for compartmentalization in droplets. For the cognate antigen, K562 cells stained with cell trace and loaded with peptide were resuspended in working buffer containing Fc block (diluted 1/5) (Miltenyi) and either anti-CD137 antibody at 2× indicated concentrations (resulting in final droplet concentrations, as indicated, respectively) or IFN-γ at a 1/25 dilution. K562 cells presenting peptide at the cell surface were pre-labeled with cell trace indicated in the figure and resuspended as defined above in medium containing X vivo with 5% human serum and supplemented with 23.6% (vol/vol)) Nycodenz, to achieve a λ (mean number of cells per droplet) of ˜0.9 for reporter cells in cell-based assays. T cells prelabeled with indicated cell trace are resuspended in the same medium mentioned above at λ˜0.45 with the required concentrations of dropcode DY754.

Microfluidic chips: Separate microfluidic chips were used. Device 1 was used to compartmentalize single cells with bioassay reagents in droplets, or to compartmentalize T cells with K562 cells presenting antigen and bioassay reagents in droplets; device 2 was used to sort droplets by fluorescence-activated dielectrophoresis; device 3 produced hydrogel beads and device 4 (CellCap) compartmentalized single sorted cells with single hydrogel beads. All chips were manufactured by soft-lithography in polydimethylsiloxane (PDMS) (Sylgard). Masters were made using one or two layers of SU-8 photoresist (MicroChem; Bear, DE), depending on the design. The list depth of the photoresist layers for devices 1 and 2 was 40 μm±1 μm and for device 3 was 55 μm±1 μm. For device 4, the first layer (70-75 μm deep) was for the hydrogel bead inlet and the second layer (130-145 μm deep) was for the cell inlet, for the reverse transcriptase inlet and for the outlet. Electrodes were prepared by melting a 51In 32.5Bi 16.5Sn alloy (Indium Corporation of America) into the electrode channel.

Droplet production, collection, and incubation: Aqueous phases I and II were co-flowed and partitioned into droplets using hydrodynamic flow-focusing in dripping mode on a microfluidic chip with a nozzle 15 μm wide, 40 μm deep, and 10 μm long. The continuous phase comprised 2-3% (wt/wt) 008-FluoroSurfactant (RAN Biotechnologies; Beverly, MA) in Novec HFE7500 fluorinated oil (3 M; Saint Paul, MN). The flow rates were adjusted to generate monodisperse droplets of 80 μl±8 μl (for cell-based assays with membrane-bound antigen). Shortly after generation, the droplets were collected into a 5 ml hemolysis tube filled with 5 ml of Novec HFE7500 fluorinated oil containing 0.1% (wt/wt) 008-FluoroSurfactant.

At least 2 emulsions were produced that contained either anti-CD137 antibody at indicated concentrations (20 nM) or anti-IFNγ detection reagent at 1/50 in droplet depending on the readout used in the droplet assay. These emulsions were differentiated using Dy754 (Dyomics) to optically encode the droplets in the positive- and negative-control emulsions and screened conditions. T cells were co-compartmentalized in droplets with K562 presenting peptide (pulsed or transfected). In certain circumstances T cells encapsulated in positive emulsion can be labelled in a different color than the T cells labelled in the negative emulsions. This is mainly used to distinguish positive from negative emulsion containing cells during flow cytometry post-QC of the enrichment of specific cells.

Microfluidic platform: Droplet fluorescence analysis and sorting was performed on a dedicated droplet microfluidic station, containing a fixed focus laser line (solid state laser of wavelength 405 nm, 488 nm, 561 nm or 635 nm, Omicron) oriented parallel to the beadline for fluorescence analysis using photomultiplier tube bandpass filters of 440/40-25 nm, 525/40-25 nm, 593/46-25 nm and 708/75-25 nm (Hamamatsu; Shizuoka, Japan).

Gating strategy for droplet sorting: The droplets were first gated to eliminate coalesced droplets and retain only droplets of the desired size. Using the optical droplet barcoding, the negative-control droplets, positive-control droplets, and droplets containing inquired cells were detected. The fluorescence relocalization to T cells was measured by plotting the maximum peak fluorescence signal (Fp) in a droplet against the integrated fluorescence signal (Fi) from the droplet. Fluorescence relocalization to T cell results in an increase in the ratio Fp/Fi. Uncoalesced droplets containing T and K562 cells were sorted if they satisfied all of the following criteria: 1) droplets containing K562 cells, 2) containing T cells, 3) relocation of the readout marker (IFN-γ and CD137) on cells within droplet and 4) colocalization of the marker fluorescence peak on T cells. The colocalization value, c, was thus bounded between 0 and 1,1 being the perfect colocalization of the two peaks. Droplets were sorted if c>0.95. For all antigens the colocalization parameter, c, was calculated from the time interval between the peaks in the fluorescence of activation marker and fluorescence of T cells, tp, and the time interval from the beginning to the end of the droplet, td: c=1−(tp/td). The value of c was thus bounded between 0 and 1,1 being the perfect colocalization of the two peaks.

Droplet sorting and cell recovery: Droplets were sorted by SAW sorter or by dielectrophoresis. This instrument can sort droplets into up to two bins, by activation of the electrodes above or beneath the channel; however, only one bin was used in this study. The inlet flows, Qem for the emulsion and Qoil for the oil, were adjusted to sort droplets at 600 s⁻¹; typical parameters for sorting were Qem=50 μl h⁻¹ (180 mbar), Qoil=50 μl h⁻¹ (180 mbar), F (the frequency of the sorting pulse)=1.5 kHz, τsort (the duration of the sorting pulse)=2,000 s and Usort (the peak-to-peak voltage applied across the electrodes)=400 kVp-p. Sorted droplets were collected in a 1.5 ml tube cooled to 4° C. Cells were recovered by addition of 100 μl of X vivo supplemented with 5% human serum, followed by 100 μl of 1H,1H,2H,2H-Perfluoro-1-octanol (Sigma, 370533; Sigma, St. Louis, MO); cells were then mixed gently and centrifuged at 300 g for 10 minutes at 4° C. to favor complete phase separation. The cells were then washed in 400 μl of 0.1% Pluronic F-68 non-ionic surfactant (Thermo Fisher Scientific, 24040032), 25 mM Hepes, 5% (vol/vol) human serum, in X vivo, centrifuged for 5 minutes at 400 g at 4° C., and were then resuspended in 50 μl of 1×PBS containing 21.82% (vol/vol) Optiprep density gradient solution (Sigma) and 0.01 mg ml-1 BSA

Production of barcoded hydrogel beads: Polyacrylamide hydrogel beads of 60-μm diameter were produced by polymerization in droplets made with a microfluidic device. However, barcoded primers were then added to the beads by split and—pool synthesis using ligation rather than primer extension. One million beads—each of which carried ˜10⁹ copies of a double stranded DNA oligonucleotide with a 5′-overhang (complementary to the first index 5′-overhang sequence), a photo-cleavable site and the T7-SBS12 sequence—were distributed into 96 wells of a microtiter plate. Each well contained 10 μl of 5 μM double-stranded DNA with a different first index (index A), a complementary 5′-overhang to the first DNA at one end and a different 5′-overhang at the other end, and these were ligated for 15 minutes at 23° C. using T7 DNA ligase (New England Biolabs) according to the manufacturer's instructions. The hydrogel beads were then pooled, washed as described and re-distributed as above into the wells of a second microtiter plate, each of which contained a double-stranded DNA with a different second index (index B), a complementary 5′-overhang to index A at one end and a different 5′-overhang at the other end, which was ligated to index A. Repetition of this splitting and pooling process 3-4 times in total (adding 3 indexes) results in 96′ combinations, which generates ˜10⁶ different barcodes. After addition of the last index, the beads were pooled, and a mixture of double-stranded DNA molecules with a complementary 5′-overhang to index C, and which contained gene-specific primer regions complementary to the regions encoding the minigenes, TCR alpha and beta and selected 20 genes, were ligated to the barcodes on the beads. The second strand of the primer was then removed by incubating for 2 minutes at 22° C. with 300 mM NaOH. After completion of the process, each hydrogel bead has a total of ˜10⁹ primers that carry the same bead-specific barcode.

Single cell barcoded complementary DNA synthesis: Individual sorted cells were co-compartmentalized in droplets with individual barcoded hydrogel beads and lysis and reverse transcription reagents using a microfluidic device. Droplets of ˜1 nl volume were formed at 250 s⁻¹. The droplets were collected in a 1.5 ml tube containing the solvent HFE-7500 (Fluorochem; Derbyshire, United Kingdom) and 0.1% surfactant, and were photo-cleaved by ultraviolet light for 90 seconds (OmniCure, AC475; 365 nm) and then incubated at 50° C. for cell lysis and cDNA synthesis

Sequencing library preparation: The emulsion containing the barcoded cDNA was broken by adding one volume of 1H,1H,2H,2H-Perfluoro-1-octanol. The pooled, barcoded cDNAs were further purified with RNA CleanXP beads (Beckman, A63987; Beckman Coulter; Brea, CA) at a 1:1 ratio (vol/vol) and eluted in 40 μl DNase- and RNase-free H₂O. The sequencing library was generated by two-step nested PCR using GoTaq Polymerase (Promega), external (PCR1) and internal (PCR2) reverse primers (SBS12 primer followed by Illumina TruSeq indexed primer-P7) and external (PCR1) and internal (PCR2) forward primers specific to TCR alpha and beta, TMG and selected genes.

Sequencing: Final products were sequenced on an Illumina (MiSeq/Nextseq), which allowed sequencing of the entire CDR3 domain of alpha and beta TCR, antigen of TMG and 20 genes as well as the barcode sequence.

### Example 1: Titration of Anti-CD137 Antibody in Droplets

In order to assess signal detection sensibility and dynamic range of immune response based on CD137 activation marker expression in the droplet, an anti-CD137 antibody titration in the droplet was performed. T cells were activated with Transact (1/100) for 48 hours at 37° C., 5% CO₂, then stained with cell trace yellow at 5 μM in PBS. Cells were then spun down at 450 g for 5 minutes and resuspended in co-flow containing complete X vivo medium with 5% human serum supplemented with Pluronic F68 at 0.10%, 23.6% of Nycodenz and 6% DNA marker (Nucgreen).

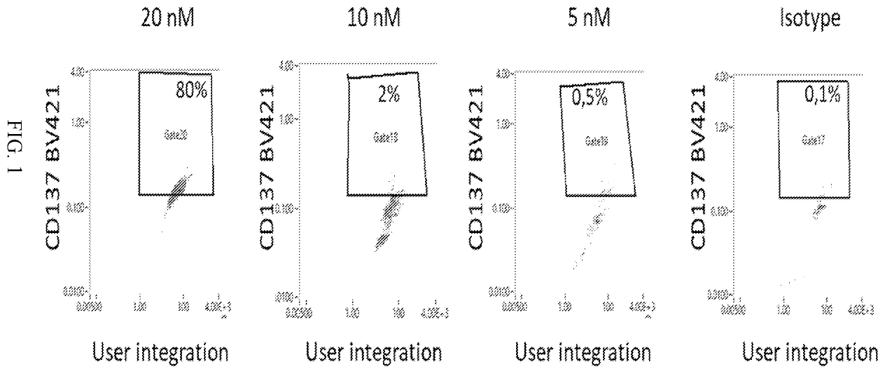

A second co-flow contained each of the different antibody concentrations tested in several emulsions assigned to different dropcode concentrations DY754 (see, Gerard et al., “High-throughput single-cell activity-based screening and sequencing of antibodies using droplet microfluidics,” Nat. Biotechnology 38:715-21 (2020)). Anti-CD137 BV421 antibody was added at twice the concentration of the co-flow to reach the indicated concentrations in the produced droplet (e.g., 5 nM, 10 nM, and 20 nM). Cells and anti-CD137 antibody were injected separately from the two different co-flow from 2 different inlets. The results of the experiment are shown in FIG. 1.

A minimum of 20 nM of anti-CD137 antibody concentration in 100 pL droplet was required to efficiently detect relocalization of the antibody at the surface of T cells, as exemplified by change of max peak detection over UserInt signal. The sensitivity of the assay was thus calculated as the minimal number of molecules to be expressed at the surface of the T cell to lead to sufficient antibody relocalization and detection of fluorescent signal. The number of calculated molecules in this assay was 120,460 molecules of CD137 per activated T cell, which would lead to 80% of activated T cell detection. Lower CD137 expression would prevent detection of activated T cells in such assay format.

### Example 2: T Cell Activation in Droplets

In order to assess efficiency of T cell activation in droplet and signal detection sensibility based on CD137 activation marker expression in the droplet, T cell stimulation by antigen presenting cells was performed and activation using anti-CD137 antibody was monitored in condition leading to efficient CD137 detection (as defined above).

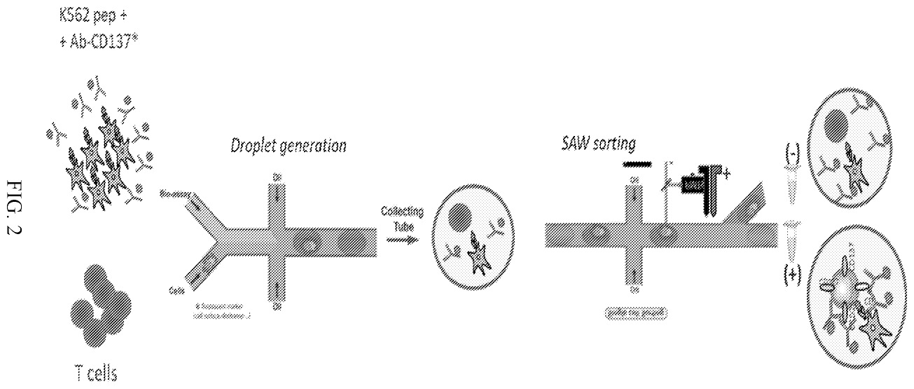

K562 cells were stained with Cell trace red at 1 μM, and then loaded with viral peptide (Epstein Barr Virus antigen BMLF1) at 10 μM. (In other experiments, the peptide can be any T cell antigen, which can include a viral antigen, a bacterial antigen, or a parasitic antigen). These cells were resuspended in co-flow containing complete X vivo medium with 5% human serum supplemented with Pluronic F68 at 0.1%, 23.6% of Nycodenz and 6% DNA marker (Nucgreen). On other hand, T cell clone specific to BMLF1 antigens were labeled with Cell trace yellow at 5 μM and suspended in similar co-flow composition as mentioned above. Anti-CD137 BV421 antibody was added at 40 nM to K562 cell suspension yielding a 20 nM droplet concentration after having co-flowed the 2 cell suspensions separately in 2 different inlets and generating therefore 100 picoliter (pl) droplet using HFE 2% surfactant. These droplets were sorted based on marker of interest expression after incubation at 37° C. and 5% CO₂ and the localization of the marker of interest in/on the T cells. FIG. 2 shows the schematic for T cell activation in the droplets.

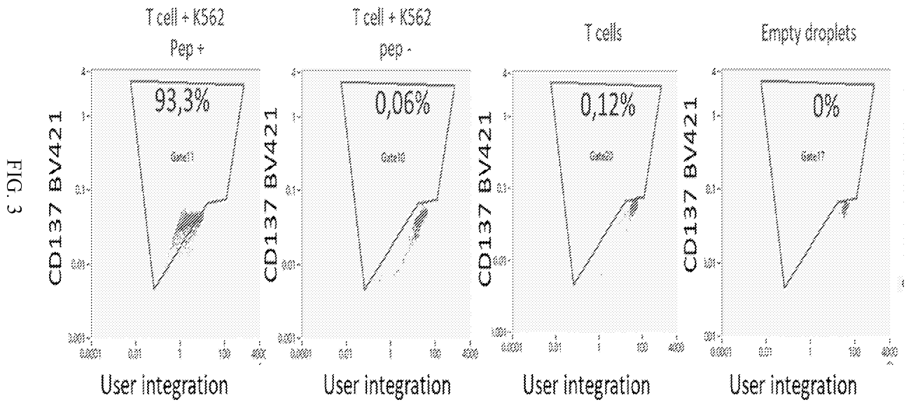

FIG. 3 shows the data gathered from the schematic approach described in FIG. 2. Briefly, 100 picoliter (pl) droplets were produced for simultaneously encapsulating the 2 cell types (i.e., a K562 cell and a T cell). These droplets were collected in a 5 ml tube containing HFE 0.1% of surfactant. This tube was incubated at 37° C. overnight. These droplets were then injected into a microfluidic device, and the droplets were examined for CD137 expression in droplets containing viable K562 and T cells, at the same time, which were labeled with different colors. User integration, a feature in the microfluidic device allowed for the estimation of area under peak, which was used to exclude any potential background and nonspecific signal (FIG. 3).

It was confirmed that efficient (˜94%) T cell activation was detected with 20 nM of CD137 antibody in the droplet using high affinity antigen and T cell clone, with minimal non-specific detection (0%-0.1%).

### Example 3: Antigen-Presenting Cell (APC)—T Cell Interactions Using Different Readout

In order to validate that the assay is specific of T and APC cell engagement/interaction in the droplet format at the single cell level, T cells stimulation was performed by antigen presenting cells in droplet and cell-cell interaction in bright field was monitored. The activation using anti-CD137 antibody, killing activity of the T cells (using NucGreen readout), and controlled specific localization were monitored for each readout.

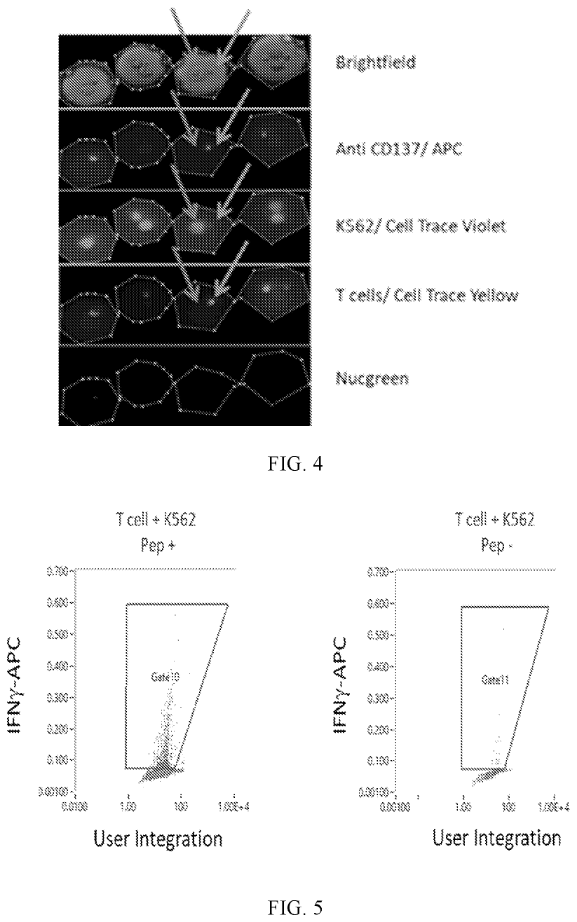

For this experiment, cells were stained in a specific fluorometric configuration to be checked under fluorescence microscopy. K562 cells were labeled with cell trace violet at 5 μM in PBS and then loaded with 10 μM peptide, while T cells were labeled in cell trace yellow at 5 μM in PBS. Both cells were resuspended in co-flow containing X vivo medium with 5% human serum supplemented with Pluronic F68 at 0.1%, 23.6% of Nycodenz and 6% DNA marker (Nucgreen). 40 nM of antibody CD137 APC were added to K562 cells co-flow while dropcode was added to T cells co-flow at desired concentration before starting droplet production. After overnight incubation at 37° C., 5% CO₂, droplets were checked under fluorescence microscopy. These images showed a clear localization of red signal (CD137) on viable T cells (yellow) and not violet (T cells) (FIG. 4). Nucgreen is a viability marker that can be used for controlling T cells killing activity (or not) coincidence (and kinetics) with activation marker co-expression.

It was confirmed that T-APC cells were engaged and interacting together in the droplet, that activated T cells produced/expressed detectable CD137 protein, as visualized by relocalization of the CD137 antibody at the surface of the T cells, and that no killing activity was detected during the course of the experiment, yet the CD137 antibody relocalization was not due to dead/dying T cells.

### Example 4: Use of Anti-IFN-γ Antibody for Detection of Activated Cells

In order to assess efficiency of T cell activation in droplet, in relevant and physiological conditions with T and APC, where APC display CMV pp65 viral antigen, and signal detection sensibility based on cytokine secretion, IFNγ activation marker expression was used as a readout. T cells stimulation was performed by antigen presenting cells and activation was monitored using anti-IFNγ antibody in condition leading to efficient IFNγ detection (not shown).

K562 cells are labeled with cell trace violet at 5 μM, and then loaded (or not loaded) with peptide at 10 μM. The K562 cells were then washed by adding cold buffer and spun down for 10 minutes at 4° C., and then the cells were resuspended in co-flow consisting of X vivo medium with 5% human serum supplemented with Pluronic F68 at 0.1%, 23.6% of Nycodenz, 6% DNA marker (Nucgreen). Anti IFN-γ detection antibody was added into this cell suspension at 1/25 dilution. For effector cells, T cells were labeled with cell Trace yellow and then with a bispecific antibody targeting CD45 and IFNγ, at 1/10 dilution in cooled medium for 5 minutes at 4° C. and at cell concentration of 10 M/ml. T cells were then washed and resuspended in co-flow containing X vivo medium with 5% human serum supplemented with Pluronic F68 at 0.1%, 23.6% of Nycodenz, 6% DNA marker (Nucgreen) and the appropriate concentration of dropcode. At the end, these cells were co-flowed separately into microfluidic device, and, thus, 100 μl droplets were produced and then incubated overnight at 37° C., 5% CO₂. Similar experiments have been done using K562 cells transfected with 10 μg of mRNA encoding the viral peptide CMV pp65 with fluorescent reporter. The results of the experiment are shown in FIG. 5.

Specific T cell activation was detected using cytokine secretion (e.g., IFNg) as the readout in the droplet using high affinity antigen and T cell clone with minimal non-specific detection.

### Example 5: TCR and Antigen Linkage Sequence Recovery from Enriched Cells Utilizing the CellCap Device

In order to recover both or either antigen and/or TCR sequence, the cells having the phenotype of interest were sorted into the microfluidic chip called CellCap. After visual inspection of the phenotype, the pair of TCR and antigen information was recovered by performing single cell barcoding using barcoded beads in the droplet containing the enriched droplet with APC expressing the antigen and the T cells. Alterative droplet recovery and sequence recovery were possible.

During cell sorting into cell cap, the library of barcoded beads prepared was washed 5 times in 5 ml 1×BW buffer (20 mM Tris-HCl Ph8.0, 50 mM NaCl, 0.1% Tween 20), and then the hydrogel beads were spun down at 3200 g for 2 minutes at 4° C. before being denaturized with 1 ml denaturation solution (970 μl H2O+30 μl 10 M NaOH, 300 mM final) for 2 minutes at room temperature (RT). Barcoded beads were then washed 3 times with 5 ml of BW buffer, and then the barcoded beads were labeled with biotinylated FITC at 5 μM final concentration at room temperature for 10 minutes. Once washed, barcoded beads were resuspended in 1 ml library buffer (Tris pH 8, 10 mM, EDTA 0.1 mM, Tween 20 0.1%), and heated for 2 minutes at 70° C. and spun down at 3200 g for 2 minutes at 4° C. The pellet was resuspended in buffer containing first strand buffer, Igepal CA_630, Sulforhodamine B and Nuclease free water) and co-flowed separately with reverse transcription (RT) mix in different inlets.

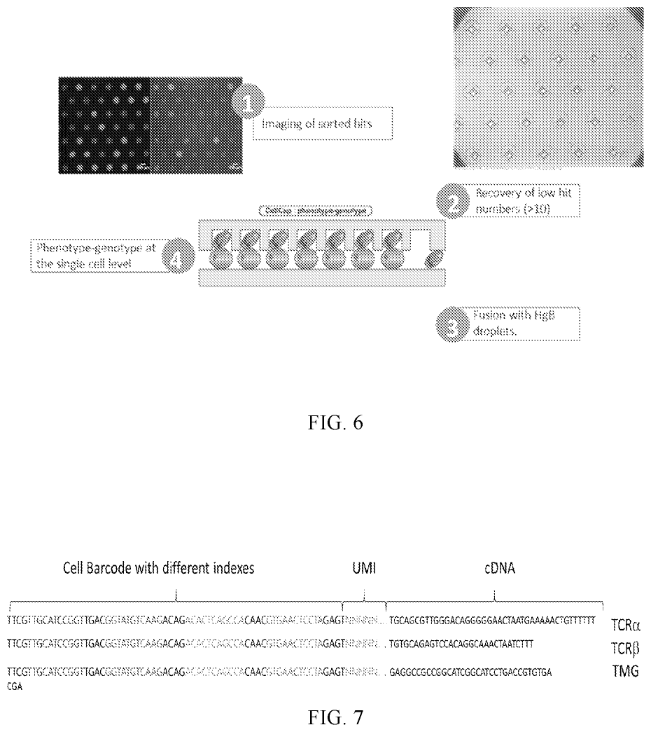

Once the CellCap was filled with 100 pl droplets containing cells, air was removed out of the chip using a syringe connected in the inlet (without chamber) while inverting CellCap reservoir. HFE was flushed at 2000 μl/hr to get completely rid of air bubbles in the system. The flow was then decreased to 150 μl/hr, and the tube containing barcoded beads emulsion was reverted until each well was filled with 1 nl droplet and all the remaining and floating droplets were flushed out of the chip. Then droplets were fused with 5*5 s and both sides (inlet and outlet were clamped) and reverse transcription (RT) was launched using a thermomixer and droplets were incubated at 50° C. for 2 hours then kept at 4° C., before inactivating the RT, processing the library preparation, and sequencing. FIG. 6 shows a schematic that is representative of the workflow.

FIG. 7 shows a representative figure of the expected sequencing data where TCR alpha and beta and antigen were recovered to identify cognate pair of receptor and ligand.

While the invention has been described in detail, and with reference to specific embodiments thereof, it will be apparent to one of ordinary skill in the art that various changes and modifications can be made therein without departing from the spirit and scope of the invention.

1. A method of identifying a cognate pair of a ligand species and a receptor species, the method comprising:
a. providing a set of ligand species, wherein each ligand species is represented at least one time;
b, providing a set of receptor species, wherein each receptor species is represented at least one time;
c. contacting the set of ligand species with the set of receptor species in a microreactor, wherein upon selective binding of a ligand species with a receptor species an enhanced signal is produced;
d. detecting a cognate pair of ligand species and receptor species by the production of the signal; and
e. identifying the cognate pair of ligand species and receptor species.

2. The method of claim 1, wherein each ligand species and/or each receptor species comprises a barcode sequence.

3. The method of claim 1, wherein each ligand species and/or each receptor species is expressed by or displayed on the surface of a cell or bead or is expressed or present in a cell free extract or in solution.

4. The method of claim 3, wherein the antigen-presenting cell is selected from a macrophage, a dendritic cell, a Langerhans cell, a B cell, a monocyte derived dendritic cell, or another cell expressing a MHC class I or II molecule.

5. The method of claim 1, wherein the microreactor is selected from an aqueous droplet, a microcapsule, a microbead, a compartment of a microfluidic chip, or a well; and/or the signal is selected from a morphological change of any one of a cell, a ligand, or a receptor; a fluorescent signal enhancement; a modification of a fluorescent signal using a caged compound or by a quenching reaction; a light absorption; a visible structure modification/creation; or a combination of signals thereof.

6. The method of claim 2, wherein identifying the cognate pair of ligand species and receptor species comprises amplifying the ligand species and/or the receptor species, wherein at least one of the amplified ligand species and receptor species are sequenced for identification.

7. The method of claim 1, wherein (a) the set of ligand species is selected from T cell antigens, B cell antigens, viral antigens, bacterial antigens, parasitic antigens, neoantigens, tumor associated antigens (TAAs), tumor specific antigens, immune checkpoint molecules, cytokines, carbohydrates, members of the immunoglobulin superfamily, selectins, chemokines, hormone, growth factors, G-protein coupled receptor ligands, or enzyme substrates; and/or (b) the set of receptor species is selected from T cell receptors, B cell receptors, immune checkpoint receptors, cytokine receptors, selectins, integrins, members of the immunoglobulin superfamily, cadherins, chemokine receptors, hormone receptors, growth factor receptors, G-protein coupled receptors (GPCRs), or enzymes.

8. The method of claim 1, wherein:
a) the ligand species is a T cell antigen and the receptor species is a T cell receptor, and upon selective binding of the T cell antigen with the T cell receptor, the enhanced signal is produced, wherein the enhanced signal produced is the result of T cell activation;
b) the ligand species is a viral antigen and the receptor species is a T cell receptor, and upon selective binding of the viral antigen with the T cell receptor, the enhanced signal is produced, wherein the enhanced signal produced is the result of T cell activation; or
c) the ligand species is a B cell antigen and the receptor species is a B cell receptor, and upon selective binding of the B cell antigen with the B cell receptor, the enhanced signal is produced.

9. The method of claim 8, wherein contacting the set of ligand species with the set of receptor species in a microreactor occurs for:
a) about 0.001 hour to about 8 hours; or
b) at least about 8 hours.

10. The method of claim 9, wherein the ligand species and the receptor species bind with high affinity and the enhanced signal produced is an early marker or late marker for T cell activation.

11. The method of claim 9, wherein the ligand species and the receptor species bind with low affinity and the enhanced signal produced is an early marker or late marker for T cell activation.

12. The method of claim 10, wherein (a) the early marker for T cell activation is selected from CD69, CD107a, or a transferrin receptor; and/or (b) the late marker for T cell activation is selected from CD137, HLA-DR, VLA1, PTA1, CD71, CD27, PD-1, TIM3, LAG3, or CTLA4.

13. The method of claim 12, wherein the enhanced signal is detected with an anti-CD69 antibody, an anti-CD107a antibody, an anti-transferrin receptor antibody, anti-CD137 antibody, an anti-HLA-DR antibody, an anti-VLA1 antibody, an anti-PTA1 antibody, an anti-CD71 antibody, an anti-CD27 antibody, an anti-PD1 antibody, an anti-TIM3 antibody, an anti-LAG3 antibody, or an anti CTLA4 antibody.

14. The method of claim 13, wherein the signal is detected with an anti-CD138 antibody, an anti-CD19 antibody, an anti-CD45R antibody, an anti-CD45 antibody, an activation of fluorescent reporter expression, or an inhibition of fluorescent reporter expression.


SEQUENCE LISTING (3 sequences)

SEQ ID NO: 1 - DNA, 111 bp, Artificial Sequence
  TCRa
  misc_feature (65)..(69): n, wherein n can be any nucleotide a, t, c, or g
ttcgttgcatccggttgacggtatgtcaagacagacactcagccacaacgtgaactcctagagtnnnnntgcagcgttgggacagggggaactaatgaaaaactgtttttt

SEQ ID NO: 2 - DNA, 99 bp, Artificial Sequence
  TCRb
  misc_feature (65)..(69): n, wherein n can be any nucleotide a, t, c, or g
ttcgttgcatccggttgacggtatgtcaagacagacactcagccacaacgtgaactcctagagtnnnnntgtgcagagtccacaggcaaactaatcttt

SEQ ID NO: 3 - DNA, 102 bp, Artificial Sequence
  TMG
  misc_feature (65)..(69): n, wherein n can be any nucleotide a, t, c, or g
ttcgttgcatccggttgacggtatgtcaagacagacactcagccacaacgtgaactcctagagtnnnnngaggccgccggcatcggcatcctgaccgtgtga